(19) United States
(12) Patent Application Publication
(10) Pub. No.: US 2023/0407307 A1
(43) Pub. Date: Dec. 21, 2023

(54) MODIFIED ANTISENSE OLIGONUCLEOTIDES TARGETING SPLICING FACTORS

(71) Applicant: The Jackson Laboratory, Bar Harbor, ME (US)

(72) Inventors: Olga Anczuków-Camarda, Bar Harbor, ME (US); Nathan Leclair, Bar Harbor, ME (US)

(73) Assignee: The Jackson Laboratory, Bar Harbor, ME (US)

(21) Appl. No.: 18/035,319

(22) PCT Filed: Nov. 4, 2021
(86) PCT No.: PCT/US2021/058010

§ 371 Date: May 4, 2023

Related U.S. Application Data
(60) Provisional application No. 63/110,225, filed on Nov. 5, 2020.

(51) Int. Cl. C12N 15/113; A61P 35/00

(52) U.S. Cl. CPC C12N 15/1135; A61P 35/00; C12N 2310/11; C12N 2310/321; C12N 2310/315; C12N 2320/33

(57) ABSTRACT

Provided herein are chemically modified antisense oligonucleotides that bind to sequences on mRNAs encoding the splicing factor TKA2β, associated with cancer.

### RELATED APPLICATION

This application claims the benefit under 35 U.S.C. § 119(e) of U.S. provisional application No. 63/110,225 filed Nov. 5, 2020, which is incorporated by reference herein in its entirety.

### GOVERNMENT LICENSE RIGHTS

This invention was made with government support under Grant No. P30 CA034196 awarded by National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Nov. 1, 2021, is named J022770095WO00-SEQ-NTJ, and is 3,576 bytes in size.

### BACKGROUND

Post-transcriptional regulation controls the expression of many genes by alternative splicing of pre-mRNA. Defects in alternative splicing are frequently found in human tumors, and RNA splicing regulatory factors have recently emerged as a new class of oncoproteins or tumor suppressors. However, there are currently no effective therapies that specifically target splicing factor defects. Most regulatory splicing factors are difficult to target by small molecule approaches because they lack a catalytic activity domain that could be inhibited by a small molecule. Thus, there is a need for alternative methods of targeting splicing factor defects for the treatment of cancer.

### SUMMARY

The present disclosure provides, in some aspects, antisense oligonucleotides (ASOs) effective for regulating alternative RNA splicing and increasing cell death in human cancer cells that overexpress TRA2β, for example. Surprisingly, the data provided herein show that the particular combination of ASO sequence and chemical modifications increased breast cancer cell death by up to 3-fold, relative to the control, an effect not observed in non-transformed (non-cancerous) human mammary epithelial cells. This effect is mediated by the ability of the chemically modified ASO to regulate alternative RNA splicing of mRNAs encoding the splicing factor TRA2β, resulting in a 60% increase in the frequency of transcripts containing a poison exon that marks the mRNA for degradation and 50% reduction in the levels of TRA2β protein expression in triple-negative breast cancer cells.

Thus, some aspects of the present disclosure provide an antisense oligonucleotide comprising a sequence that binds to a target sequence on an mRNA that encodes TRA2β, wherein the antisense oligonucleotide further comprises a 2′-O-methoxyethyl modification. In some embodiments, at least 80% (e.g., at least 85%, at least 90%, at least 95%) of the nucleotides of the antisense oligonucleotide comprise a 2′-O-methoxyethyl modification. In some embodiments, each of the nucleotides of the antisense oligonucleotide comprise a 2′-O-methoxyethyl modification.

In some embodiments, the antisense oligonucleotide further comprises a phosphorothioate modification. In some embodiments, at least 80% of the internucleoside phosphates of the antisense oligonucleotide comprise a phosphorothioate modification. In some embodiments, each of the internucleoside phosphates of the antisense oligonucleotide comprises a phosphorothioate modification. In some embodiments, each of the nucleotides of the antisense oligonucleotide comprises a 2′-O-methoxyethyl modification, and each of the internucleoside phosphates of the antisense oligonucleotide comprises a phosphorothioate modification.

In some embodiments, the target sequence is within an intronic splicing silencer sequence. In some embodiments, binding of the antisense oligonucleotide increases TRA2β-PE inclusion and/or decreases TRA2β protein expression by at least 30%, at least 40%, at least 50%, at least 60%, at least 70%, at least 80%, or at least 90% relative to a non-targeting control antisense oligonucleotide.

In some embodiments, the target sequence comprises a sequence having at least 50%, at least 60%, at least 70%, at least 80%, or at least 90% sequence identity to the sequence of SEQ ID NO: 2 (e.g., across at least 50%, at least 60%, at least 70%, at least 80%, at least 90%, or at least 100% of the length of the sequence of SEQ ID NO: 2). For example, the target sequence comprises a sequence having at least 95% sequence identity to the sequence of SEQ ID NO: 2. In some embodiments, the target sequence comprises the sequence of SEQ ID NO: 2.

In some embodiments, the target sequence comprises a sequence having at least 50%, at least 60%, at least 70%, at least 80%, or at least 90% sequence identity to the sequence of SEQ ID NO: 3 (e.g., across at least 50%, at least 60%, at least 70%, at least 80%, at least 90%, or at least 100% of the length of the sequence of SEQ ID NO: 3). For example, the oligonucleotide comprises a sequence having at least 95% sequence identity to the sequence of SEQ ID NO: 3. In some embodiments, the oligonucleotide comprises the sequence of SEQ ID NO: 3.

In some embodiments, no more than one nucleotide substitution relative to the sequence of SEQ ID NO: 3. In some embodiments, no more than two nucleotide substitutions relative to the sequence of SEQ ID NO: 3.

In some embodiments, the ASO is formulated in a delivery vehicle. In some embodiments, the delivery vehicle is a cationic lipid nanoparticle. In some embodiments, the delivery vehicle is a liposome.

Other aspects of the present disclosure provide a delivery vector comprising the antisense oligonucleotide of any one of the preceding paragraphs. In some embodiments, the delivery vector is a viral vector.

The present disclosure further provides, in some aspects, a host cell comprising the antisense oligonucleotide of any one of the preceding paragraphs or the delivery vector of the preceding paragraph. The host cell may be, for example, a cancer cells, such as a breast cancer cell (e.g., a triple-negative breast cancer cell), an ovarian cancer cell, a colon cancer cell, a glioblastoma cell, a bladder cancer cell, a kidney cancer cell, a liver cancer cell, a lung cancer cell, or a prostate cancer cell.

Yet other aspects of the present disclosure provide a pharmaceutical composition comprising the antisense oligonucleotide of any one of the preceding paragraphs, or the delivery vector, and a pharmaceutically acceptable excipient.

Further aspects of the present disclosure provide a method comprising administering to a subject the antisense oligonucleotide or the pharmaceutical composition.

In some embodiments, the subject has breast cancer, ovarian cancer, colon cancer, glioblastoma, bladder cancer, kidney cancer, liver cancer, lung cancer, or prostate cancer. In some embodiments, the subject has breast cancer. For example, the subject may have triple-negative breast cancer.

In some embodiments, the administering is intravenous, intramuscular, intraperitoneal, subcutaneous, intranasal, or intratumoral.

Also provided herein, in some aspects, is a method comprising synthesizing the antisense oligonucleotide of any one of the preceding paragraphs.

Further provided herein, in some aspects, is a method comprising formulating the antisense oligonucleotide of any one of the preceding paragraphs with a pharmaceutically acceptable excipient.

The present disclosure also provides, in some aspects, a kit comprising the antisense oligonucleotide or the pharmaceutical composition of any one of the preceding paragraphs and a delivery device.

### BRIEF DESCRIPTION OF THE DRAWINGS

FIGS. 1A-1G. Splice-switching antisense oligonucleotides promote TRA2β-PE inclusion, leading to decreased TRA2β protein levels and activity and increased breast cancer cell death. (FIG. 1A) Principle of using splice-switching antisense oligonucleotides (ASOs) to block intronic silencer sequences (ISS) and promote TRA2β-PE inclusion. (FIG. 1B) TRA2β-PE splicing in MDA-MB231 and SUM159 breast cancer cells transfected with 50 nM of TRA2β-targeting (1570) or non-targeting control (CTL) 2′-O-methoxyethyl-phosphorothioated (2′MOE) ASOs. TRA2β-PE inclusion and TRA2β protein expression are measured with RT-PCR and western blot, respectively, 48 h after transfection (n=3, mean±SD; t-test, *P<0.05, **P<0.01). (FIG. 1C) Cell proliferation in MDA-MB231 and SUM159 breast cancer cells transfected with 50 nM of 2′MOE ASO-1570 vs. ASO-CTL is quantified by 5-Ethynyl-2′-deoxyuridine (EdU) incorporation 48 h after transfection. Representative stains for Alexa 647 EdU and Hoechst in MDA-MB231 cells are shown in the left panel. Proliferation is quantified as the percent of EdU+ cells to total Hoechst+ cells (n=3, mean±SD; t-test, *P<0.05, **P<0.01). (FIG. 1D) Cell death in MDA-MB231 and SUM159 breast cancer cells transfected with 50 nM of 2′MOE ASO-1570 vs. -CTL is quantified by AnnexinV staining 48 h after transfection. Representative stains for Alexa 647 conjugated AnnexinV and Hoechst in MDA-MB231 and SUM159 cells are shown in the left panel. The percent of AnnexinV+ cells to total Hoechst+ cells is normalized to ASO-CTL (n=3, mean±SD; t-test, *P<0.05, **P<0.01). (FIG. 1E) Splicing of known TRA2β target transcripts is measured in MDA-MB231 and SUM159 cells transfected with 50 nM of 2′MOE ASO-1570 vs. ASO-CTL, 48 h after ASO transfection. Exon inclusion is measured by RT-PCR (n=3, mean±SD; t-test,*P<0.05, **P<0.01). (FIG. 1F) TRA2β-PE splicing in MCF-10A and AR7-HMEC non-transformed mammary epithelial cells transfected with 50 nM of TRA2β-targeting (1570) or non-targeting control (CTL) 2′-O-methoxyethyl-phosphorothioated (2′MOE) ASOs. TRA2β-PE inclusion and TRA2β protein expression are measured with RT-PCR and western blot, respectively, 48 h after transfection (n=3, mean±SD; t-test, *P<0.05, **P<0.01). (FIG. 1G) Cell death in MCF-10A and AR7-HMEC non-transformed mammary epithelial cells transfected with 50 nM of 2′MOE ASO-1570 vs. -CTL is quantified by AnnexinV staining 48 h after transfection. Representative stains for Alexa 647 conjugated AnnexinV and Hoechst in MCF-10A and AR7-HMEC cells are shown in the left panel. The percent of AnnexinV+ cells to total Hoechst+ cells is normalized to ASO-CTL (n=3, mean±SD; t-test, *P<0.05, **P<0.01).

FIGS. 2A-2L. TRA2β-targeting ASOs impact TRA2β-PE splicing as well as TRA2β protein expression and activity. (FIG. 2A) Binding locations of ASOs targeting TRA2β-PE and the downstream intron. The bottom panel shows the binding sequence of ASO-1570 along with the location of 423 deletion, as well as the splicing silencer and HNRNPA1 binding motifs predicted by Human Splicing Finder (Desmet et al., 2009). (FIG. 2B) TRA2β-PE splicing in MDA-MB231 cells transfected with 50 nM of TRA2β-targeting or non-targeting control (CTL) 2′-O-methyl-phosphorothioated (2′OMe) ASOs. TRA2β-PE inclusion and TRA2β protein expression are measured with RT-PCR and western blot, respectively, 48 h after transfection (n=3, mean±SD; t-test, *P<0.05, **P<0.01). (FIG. 2C) TRA2β-PE splicing in HS578T cells transfected with 50 nM of TRA2β-targeting or non-targeting control (CTL) 2′-O-methyl-phosphorothioated (2′OMe) ASOs. TRA2β-PE inclusion and TRA2β protein expression are measured with RT-PCR and western blot, respectively, 48 h after transfection (n=3, mean±SD; t-test, *P<0.05, **P<0.01). (FIG. 2D) HeLa cells cotransfected with the TRA2β-PE minigene and either a control plasmid (CTL) or one encoding an HA-tagged hnRNPA1-CDS. Minigene splicing and protein production assessed by RT-PCR and western blotting 48 h after transfection (n=3, mean±SD; t-test, *P<0.05, **P<0.01). (FIG. 2E) HeLa cells cotransfected with the TRA2β-PE minigene along with TRA2β-targeting (1570) or control (CTL) ASO alone, or with increasing amounts of HA-hnRNPA1-CDS or control plasmids. Minigene splicing and protein production assessed by RT-PCR and western blotting 48 h after transfection, compared to ASO-1570 alone. (FIG. 2F) TRA2β-PE splicing and TRA2β protein expression in HS578T cells transfected with 50 nM of ASO-1570 (n=3, mean±SD; t-test, *P<0.05, **P<0.01). (FIG. 2G) Cell proliferation in HS578T breast cancer cells transfected with 50 nM of 2′MOE ASO-1570 vs. -CTL is quantified by 5-Ethynyl-2′-deoxyuridine (EdU) incorporation 48 h after transfection. Representative stains for Alexa 647 EdU and Hoechst in MDA-MB231 cells are shown in the left panel. The percent of EdU+ cells to total Hoechst+ cells is shown (n=3, mean±SD; t-test, *P<0.05, **P<0.01). (FIG. 2H) Cell death in HS578T breast cancer cells transfected with 50 nM of 2′MOE ASO-1570 vs. -CTL is quantified by AnnexinV staining 48 h after transfection. Representative stains for Alexa 647 conjugated AnnexinV and Hoechst in MDA-MB231 and SUM159 cells are shown in the left panel. The percent of AnnexinV+ cells to total Hoechst+ cells is normalized to ASO-CTL (n=3, mean±SD; t-test, *P<0.05, **P<0.01). (FIG. 2I) Wound closure assays in HS578T and SUM159 cells transfected with 50 nM of 2′MOE ASO-1570 vs. -CTL. Percent wound closure is relative to 0 h (n=3, mean±SD; t-test,*P<0.05, **P<0.01). (FIG. 2J) Splicing of known TRA2β target transcripts is measured in HS578T cells transfected with of 2′-O-methoxyethyl-phosphorothioated (2′MOE) ASO-1570 vs. -CTL, 48 h after ASO transfection. Exon inclusion is measured by RT-PCR (n=3, mean±SD; t-test *P<0.05, **P<0.01). (FIG. 2K) Wound closure assays in MCF-10A and AR7-HMEC non-transformed mammary epithelial cells transfected with 50 nM of 2′MOE ASO-1570 vs. -CTL. Percent wound closure is relative to 0 h (n=3, mean±SD; t-test,*P<0.05, **P<0.01). (FIG. 2L) GFP-tagged MDA-MB231 co-cultured with MCF-10A cells are transfected with 50 nM of 2′MOE ASO-1570 vs. -CTL. Cell number is quantified at various timepoints by live cell imaging with GFP and Hoechst stain. The proportion of MDA-MB231 cells is calculated as number of GFP+ cells divided by total number of Hoechst+ nuclei (n=3, mean±SD; t-test, *P<0.05, **P<0.01).

FIGS. 3A-3C. TRA2β-PE inclusion is regulated by discrete exonic and intronic sequences contained within its ultraconserved region. (FIG. 3A) Location of −25 bp deletions (A1 to A30) spanning the exon and surrounding introns in mutant TRA2β-PE minigenes are shown schematically (grey boxes) in the top panel, along with the location of the ultraconserved region (UCR). Wild-type and mutant TRA2β-PE minigenes are transfected into HeLa cells. TRA2β-PE inclusion is quantified by RT-PCR with minigene-specific primers and normalized as APSI to wild type minigene (n=3, mean±SD; t-test, *P<0.05, **P<0.01). Log 2 fold change (FC) protein expression is quantified by western blotting using a minigene-specific Myc-tag antibody and tubulin as a loading control, normalized to wild type minigene (n=3, mean±SD; t-test, *P<0.05, **P<0.01). (FIG. 3B) Binding positions of 22 nt gRNAs tilled across the exon (e2-1 to -24), upstream intron (i1-1 to -8) or downstream intron (i2-1 to -8) are shown in the top panel. HEK293T cells are transfected with the wild-type TRA2β-PE minigene along with dCasRx and gRNA encoding plasmids. TRA2β-PE inclusion is assessed by RT-PCR with minigene specific primers and normalized as APSI to non-targeting control gRNA (CTL). (FIG. 3C) Overlap of the sequence deletion locations from (FIG. 3A) and dCasRx gRNAs binding positions from (FIG. 3B) along with their effects on TRA2β-PE inclusion (light gray for inclusion, dark gray for skipping) are shown in the top panel. Position of RBP motifs predicted by RBPmap in two regions of interest are indicated on the bottom panel along with deletion mutants and gRNAs positions.

FIG. 4. Decreased TRA2β poison exon inclusion correlates with lower patient survival across multiple cancer types. TRA2β-PE inclusion is quantified in TCGA tumors and z-scored by tumor type. Patients are stratified by low (z-score <0.5) and other (z-score >0.5) TRA2β-PE inclusion levels. Patient survival (Liu et al., Cell 2018) based on TRA2β-PE is calculated using survival and survminer packages.

FIG. 5. ASO-1570 increases poison exon inclusion and decreases TRA2β protein levels across multiple cancer cell lines. TRA2β exon inclusion as percent spliced in (PSI) is measured by quantitative RT-PCR in cancer cells transfected with 200 nM of control (CTL) or TRA2β-targeting (−1570 or -in2-8) 2′MOE ASOs, normalized as APSI to ASO-CTL (n=3). TRA2β protein levels are measured by western blot, normalized to actin loading control and as fold change to ASO-CTL (n=3; mean±stdev; t-test; ns not significant, *P<0.05, **P, 0.01).

FIGS. 6A-6C. ASO-1570 promotes cell death and decreases cell proliferation by modulating poison exon inclusion and TRA2β protein levels in glioblastoma U87-MG cells. (FIG. 6A) TRA2β exon inclusion as percent spliced in (PSI) is measured by quantitative RT-PCR in U87-MG cancer cells transfected with CTL or TRA2β-targeting 2′MOE ASO-1570 or in2-8, normalized as APSI to ASO-CTL (n=3). TRA2β protein levels are measured by western blot, normalized to actin loading control and as fold change to ASO-CTL (n=3; mean±stdev; t-test). (FIG. 6B) Cell death in U87-MG cancer cells transfected with 200 nM of 2′MOE ASO-1570 vs. -CTL is quantified by Cell Event Caspase-3/7 detection reagent 48 h after transfection. Representative caspase activity and Hoechst are shown. (FIG. 6C) Cell proliferation in U87-MG cancer cells transfected with 200 nM of 2′MOE ASO-1570 vs. -CTL is quantified by 5-Ethynyl-2′-deoxyuridine (EdU) incorporation 48 h after transfection. Representative stains for Alexa 647 EdU and Hoechst are shown.

FIG. 7. ASO-1570 exhibits pro-tumorigenic activity across multiple cancer subtypes by modulation poison-exon splicing. Indicated cancer cells (left panel) are transfected with 2′MOE ASO-CTL, -1570, or -in2-8 at indicated concentrations (from 25 nM to 200 nM). At 48 h after transfection, changes in TRA2β splicing and protein levels are measured by quantitative RT-PCR and western blot respectively, and plotted for ASO-1570, or -in2-8 normalized to ASO-CTL (n=3, mean±SD; t-test, *P<0.05, **P<0.01). Cell death and cell proliferation are assayed by AnnexinV/Hoechst staining and EdU/Hoechst staining respectively, and plotted for ASO-1570, or -in2-8 normalized to ASO-CTL (n=3, mean±SD; t-test, *P<0.05, **P<0.01).

FIG. 8. TRA2β protein knockdown is not sufficient to induce cell death or decreased proliferation across multiple cancer subtypes. Indicated cancer cells (left panel) are transfected with CTL or TRA2β-targeting siRNAs. At 48 h after transfection, changes in TRA2β splicing and protein levels are measured by semi-quantitative PCR and western blot, respectively, and plotted for TRA2β-targeting to CTL (n=3, mean±SD; t-test, *P<0.05, **P<0.01). Cell death and cell proliferation are assayed by Caspase3/7/Hoechst staining and EdU/Hoechst staining, respectively, and plotted for TRA2β-targeting normalized to CTL (n=3, mean±SD; t-test, *P<0.05, **P<0.01).

FIGS. 9A-9B. TRA2β poison exon-containing transcript localizes to the nucleus. (FIG. 9A) Localization of all TRA2β transcripts (total) or only the TRA2β poison exon-containing transcript in cells transfected 2′MOE ASO-CTL or ASO-1570 labelled using exon-specific RNA FISH using probes. (FIG. 9B) Localization of the Alexa568-labeled ASO ASO-1570. Nuclei are stained with DAPI.

FIGS. 10A-10D. ASO-1570 causes significant changes in RNA isoform expression in cancer cell lines. RNA-sequencing data from MDA-MB231 and U87-MG cells was analyzed for differential splicing events between treatment with either 100 nM of 2′MOE control ASO (ASO-CTL) or 2′MOE ASO-1570. (FIG. 10A) Number and event type proportion of significantly splicing events between ASO-1570 and ASO-CTL detected by rMATS. (FIG. 10B) Histogram of significantly splicing events between ASO-1570 and ASO-CTL demonstrating range of inclusion and skipping events. (FIG. 10C) Overlap of differential splicing events between MDA-MB231 and U87-MG cell lines. (FIG. 10D) Heatmap of PSI values (z-score) of the 4376 overlapping splicing events.

### DETAILED DESCRIPTION

Regulation of splicing-factor (SF) expression determines the balance of different RNA isoforms within a cell, and dysregulation of SF levels is associated with diseases such as cancer. SFs contain ultraconserved poison exon (PE) sequences, which exhibit greater identity across species than nearby coding exons, yet their physiological role and molecular regulation is incompletely understood. In the course of studying how splicing factors are regulated at the post-transcriptional level, experimental data revealed that serine/arginine-rich (SR) splicing factors cross-regulate their mRNA, and therefore protein expression, by using alternative splicing coupled to nonsense-mediated mRNA decay, and that this regulation is altered in tumors compared to normal tissues. It was therefore postulated that targeting this post-transcriptional regulatory mechanism could manipulate SR protein levels and serve as an anticancer therapy.

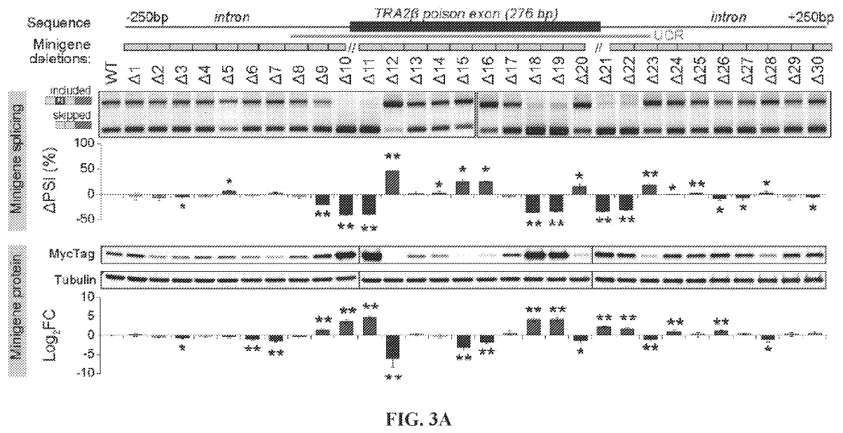

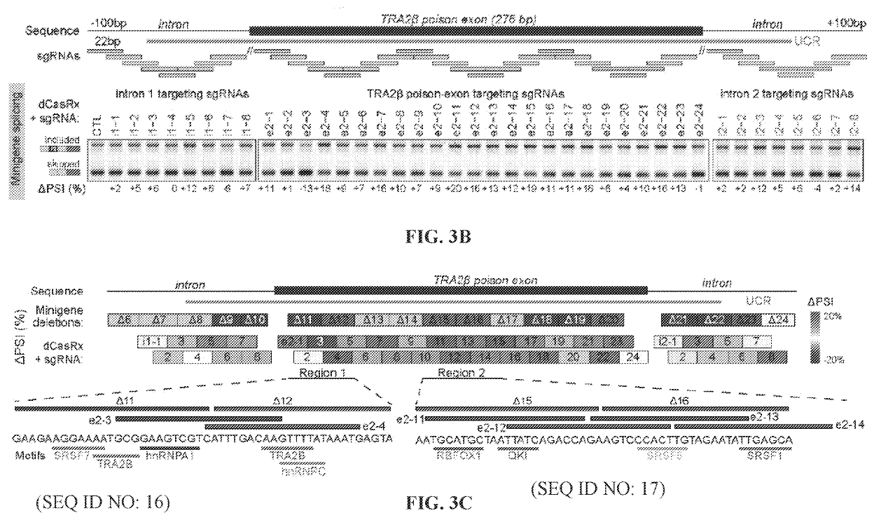

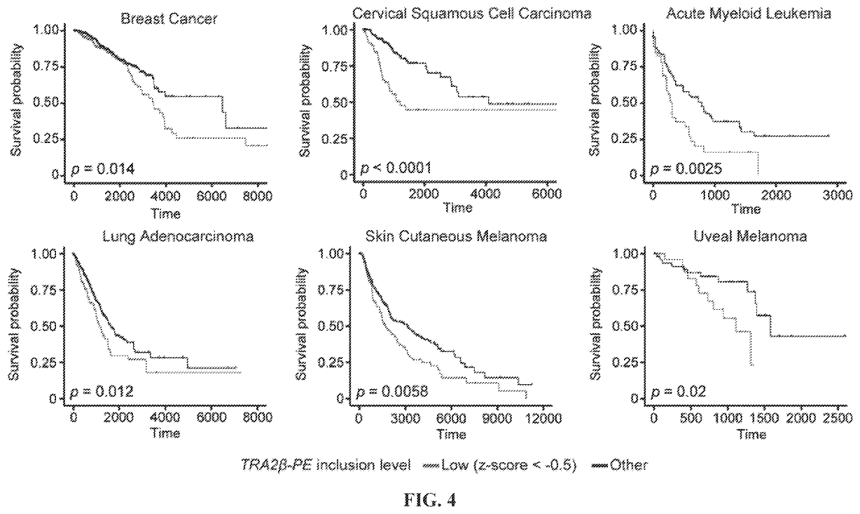

Initial studies focused on TRA2β, an oncogenic splicing factor upregulated and/or amplified in 13% of human breast tumors, half of which are classified as triple-negative breast cancer, an aggressive tumor subtype associated with poor prognosis and a lack of efficient therapies. Using minigene deletion experiments, as well as a CRISPR screen using catalytically inactive Cas9, key regions that control TRA2β splicing, and therefore its protein levels, were identified (FIGS. 3A-3C). These regions were investigated further as potential therapeutic targets.

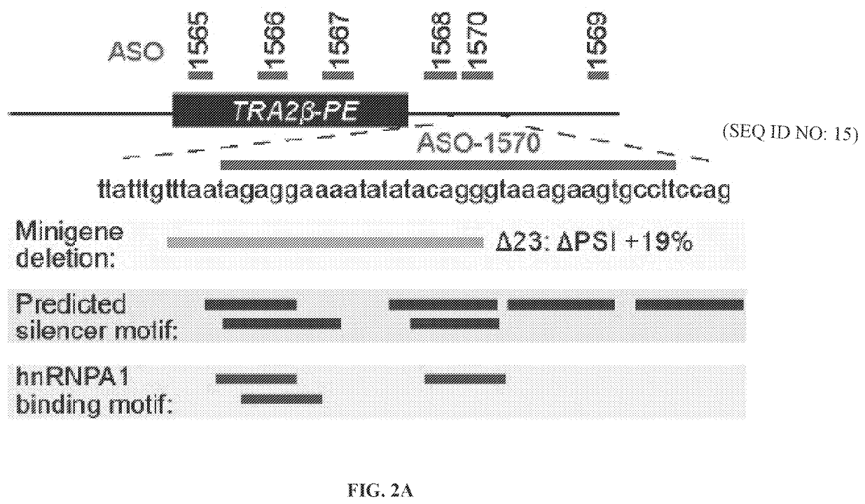

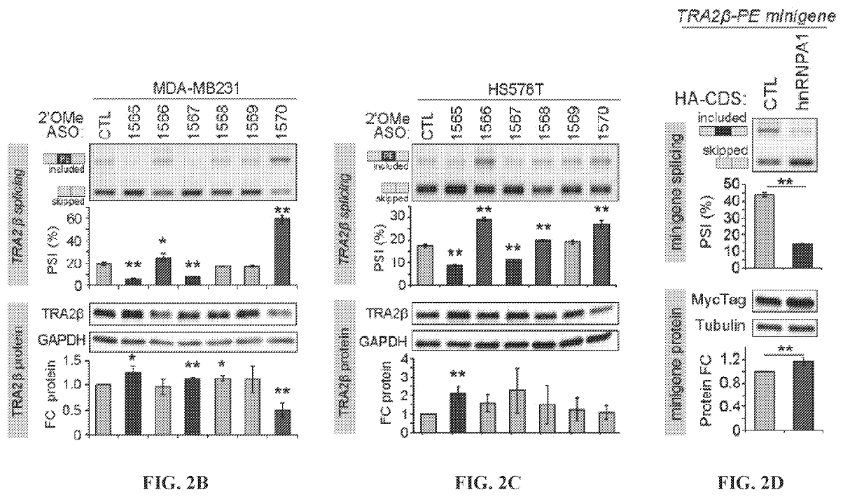

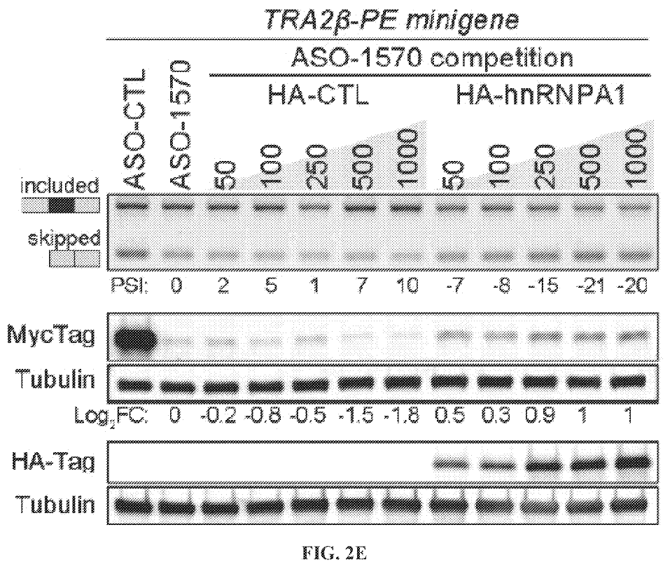

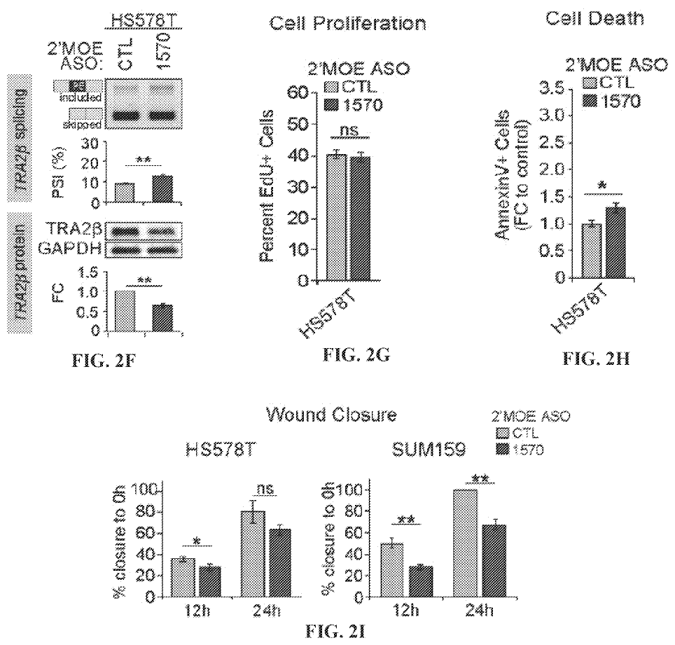

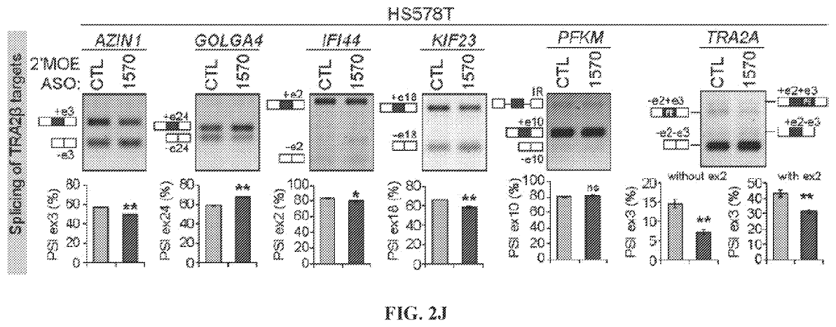

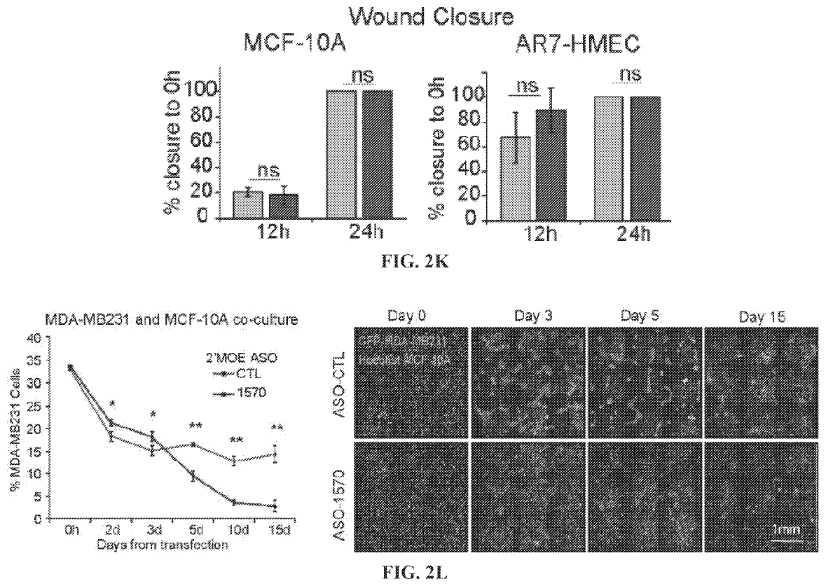

Splice-switching antisense oligonucleotides (ASOs) complementary to these regions were designed and tested to evaluate their effects on splicing and protein levels. Twelve (12) ASOs with sequences targeting key regulatory regions, as well as 2′-O-methoxyethyl (2′MOE) and phosphorothioate (PS) modifications to the backbone, were tested for their effects on splicing in a minigene. Six (6) additional ASOs containing different sequences, as well as 2′-O-methyl (2′OMe) and PS backbone modifications, but not 2′MOE modifications, were also tested for their effects on splicing in minigene experiments (FIGS. 2A-2C). One of these ASOs, referred to herein as 2′OMe ASO-1570, had the strongest effect on the minigene, but a limited effect on the expression of TRA2β protein in certain cell lines and was highly toxic to the cells (FIGS. 2A-2C). Surprisingly, however, when the ASO-1570 sequence was redesigned to omit the 2′OMe and instead include the 2′MOE modification (referred to herein as 2′MOE ASO-1570), the redesigned ASO effectively suppressed endogenous protein expression in all cell lines tested and cell toxicity was eliminated.

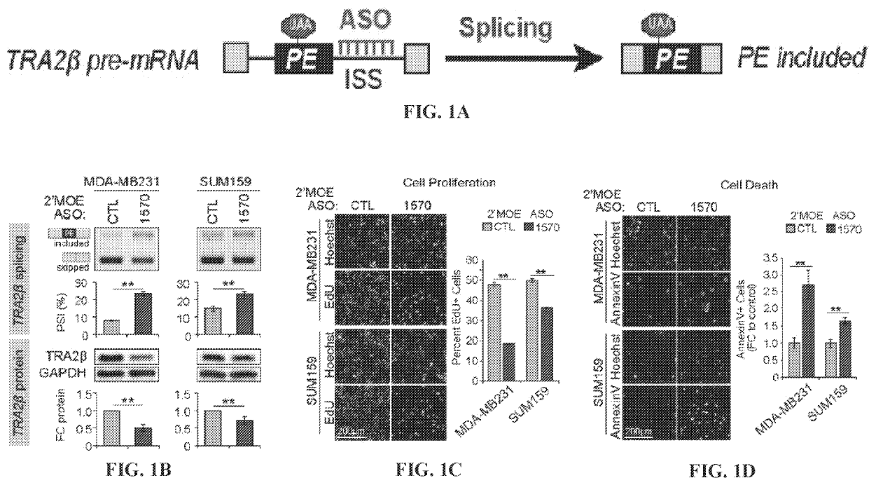

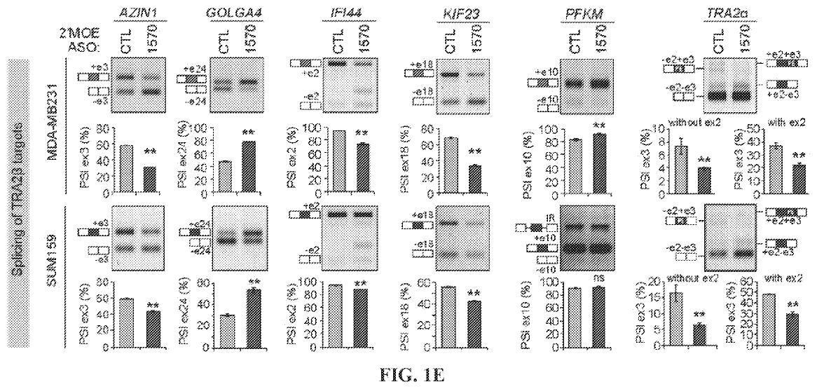

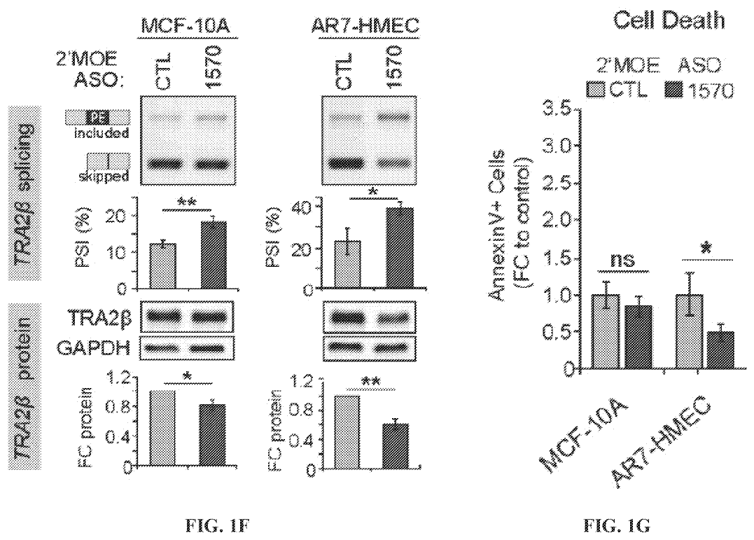

Unexpectedly, this 2′MOE chemical modification to the ASO-1570 backbone markedly improved its activity in cancer cells compared to an ASO of the same sequence but with 2′OMe (instead of 2′MOE) backbone modifications. Specifically, when transfected into human triple-negative HS578T breast cancer cells, 2′OMe ASO-1570 exhibited no change in TRA2β protein expression (FIG. 2C), but transfection of the same cells with 2′MOE ASO-1570 significantly reduced protein expression (FIG. 2G). The results of experiments with 2′MOE ASO-1570 are shown in FIGS. 1A-1G and 2A-2L.

Transfection with 2′MOE ASO-1570 increased TRA2J3 poison-exon inclusion, and therefore decreased protein levels, relative to a control ASO with similar backbone modifications but a non-targeting sequence, in both human triple-negative breast cancer cell lines and non-transformed human mammary epithelial cells (FIGS. 1B and 1F). Furthermore, transfection with 2′MOE ASO-1570 increased cell death in human triple-negative breast cancer cell lines, but not in non-transformed human mammary epithelial cells (FIG. 1G).

These results demonstrate that 2′MOE ASO-1570 is an effective negative regulator of TRA2β mRNA and protein expression, and thus is a promising therapy for triple-negative breast cancer.

### Antisense Oligonucleotides

The present disclosure provides antisense oligonucleotides (ASOs) comprising a sequence that binds to a target sequence on an mRNA encoding TRA2β. An ASO is a single-stranded DNA or RNA oligonucleotide that is complementary to a chosen target sequence. The terms oligonucleotide and nucleic acid can be used interchangeably referring to at least two nucleotides and/or nucleotide analogs covalently linked together, and in some instances, containing phosphodiester bonds, referred to as a phosphodiester backbone. For the purpose of clarity, the therapeutic molecules described herein are referred to as oligonucleotides, while the targets of interest are referred to in the context of a nucleic acid. Thus, an antisense oligonucleotide binds to a target nucleic acid.

An oligonucleotide is complementary to a chosen target sequence if the oligonucleotide binds to a nucleic acid comprising the target sequence, forming a nucleic acid that is at least partially double-stranded through hydrogen bonds between base pairs on the oligonucleotide and target sequence. An oligonucleotide is most complementary to a sequence when the oligonucleotide comprises a sequence of bases that form canonical Watson-Crick base pairs (i.e., A-U, A-T, C-G) with the target sequence, in reverse order relative to the order of bases in the target sequence. An oligonucleotide with this sequence of complementary bases in reverse order is said to have the reverse complement of the target sequence. For example, the reverse complement of the target sequence AAGUCCA is TGGACTT (DNA) or UGGACUU (RNA). An antisense oligonucleotide may still bind to a target sequence even if the sequence of the antisense oligonucleotide differs from the exact reverse complement of the target sequence by one or more nucleotides, provided the sequence of the antisense oligonucleotide is sufficiently similar to the reverse complement of the target sequence. The exact level of sequence identity between the sequence of an antisense oligonucleotide and the reverse complement of the target sequence that is sufficient for an antisense oligonucleotide to bind to a given target sequence will depend on the sequences of the antisense oligonucleotide and target sequence, for example, the nucleotide composition and/or length, as well as the binding conditions (e.g., in vivo human physiological conditions). Methods of determining whether an antisense oligonucleotide comprising a given sequence binds to a nucleic acid comprising a target sequence are well known in the art.

Binding of an antisense oligonucleotide to an mRNA target can interfere with normal cellular processing of the mRNA, and therefore expression of the encoded gene, through multiple mechanisms. For example, nucleases of the ribonuclease H (RNase H or RNH) family hydrolyze the phosphodiester bond between nucleotides in the RNA component of a DNA/RNA hybrid nucleic acid, in which a single-stranded DNA sequence is hybridized with a single-stranded RNA sequence. An mRNA bound by an antisense DNA oligonucleotide may thus be cleaved by RNase H, thereby preventing translation of the mRNA into an encoded protein. ASOs may also modulate gene expression by interfering with the formation of a 5′ cap on mRNA, altering the splicing process (splice-switching), and hindering translation by ribosomes through steric hindrance.

The present disclosure, in some aspects, provides methods of synthesizing antisense oligonucleotides comprising a given sequence. Synthesizing refers to the process of oligonucleotide synthesis, a chemical process in which oligonucleotides with defined chemical structures, or sequences, are produced. Rather than nucleotides of the canonical nucleotide structure, comprising a ribose or deoxyribose sugar linked to a phosphate group and purine or pyrimidine base, the individual molecules used in oligonucleotide synthesis are nucleoside phosphoramidites. Nucleotide phosphoramidites differ from canonical nucleotides in that certain moieties are protected to reduce their reactivity. These modifications include a 4,4′-dimethoxytrityl (DMT) group bound to the oxygen atom of the 5′ hydroxyl group of the ribose or deoxyribose, a 2-cyanoethyl group bound to an oxygen atom of the phosphate group, and an amine group bound to the phosphorus atom of the phosphate group. In some embodiments, the constituent oligonucleoside phosphoramides contain further chemical modifications, such as phosphorothioate (PS), 2′-O-methyl (2′OMe), or 2′-O-methoxyethyl (2′MOE) modifications, which are described in more detail in the following section.

Oligonucleotide synthesis is well understood in the art and is typically carried out by immobilizing the first nucleoside phosphoramide of a desired sequence to a solid support, such as controlled pore glass or microporous polystyrene. A second nucleotide is then added to the 5′ hydroxyl group of the first nucleotide by a chemical reaction comprising four steps. First, the protective moiety of the first nucleotide is removed, in a process known as detritylation. Second, the second nucleotide is coupled to the first, in a reaction joining the phosphate group of the second nucleotide to the hydroxyl group of the first nucleotide, resulting in an oligonucleotide containing two nucleotides. Third, a capping step is performed by treating the solid support-bound material with a mixture of acetic anhydride and 1-methylimidazole, which acetylates the 5′ hydroxyl groups of any first nucleotides that were not joined to a second nucleotide, thereby preventing them from reacting with nucleotides that are added in future steps. Fourth, the support-bound material is treated with iodine and water in the presence of a weak base to oxidize the phosphite triester. Synthesis then proceeds by repeating these four steps, resulting in stepwise addition of individual nucleotides to the terminus of the growing oligonucleotide. After the addition of the final nucleotide in the desired sequence, the oligonucleotide is treated with a mixture comprising aqueous ammonium hydroxide, aqueous methylamine, gaseous ammonia, or gaseous methylamine, removing all remaining protecting groups. The resulting oligonucleotide may then be purified by polyacrylamide gel electrophoresis or anion-exchange high performance liquid chromatography followed by desalting. Other synthesis methods may be used.

### Chemical Modifications

The antisense oligonucleotides provided herein comprise a chemical modification. A chemical modification is a change in the structure of a molecule that differentiates it from its native structure. With respect to nucleotides and oligonucleotides, native structure refers to the structures of ribose, deoxyribose, phosphate, RNA, and DNA that are well understood in the art.

In some embodiments, the antisense oligonucleotides comprise a phosphorothioate (PS) modification. A phosphorothioate modification is a chemical modification in which a sulfur atom replaces one of the non-bridging oxygen atoms (i.e., one of the oxygen atoms that is not bound to both a phosphorus atom and a carbon atom of a sugar) in the internucleoside phosphate group of a nucleotide or the sugar-phosphate backbone of a nucleic acid. A nucleic acid in which the phosphate groups contain phosphorothioate modifications is said to have phosphorothioate bonds, PS bonds, and/or a PTO backbone. ASOs containing PS modifications exhibit improved nuclease resistance, making them less likely to be degraded before they can hybridize to their target sequence, but are less specific than unmodified ASOs of the same sequence, and may thus cause cytotoxicity when administered at high doses due to binding to sequences other than their target. In some embodiments, at least 50%, at least 60%, at least 70%, at least 80%, at least 90%, at least 95%, at least 97%, at least 98%, or at least 99% of the nucleotides of the antisense oligonucleotide comprise a phosphorothioate modification. In some embodiments, each of the nucleotides of the antisense oligonucleotide comprise a phosphorothioate modification.

In some embodiments, the antisense oligonucleotides comprise a 2′-O-methoxyethyl (2′MOE) modification. A 2′-O-methoxyethyl modification is a chemical modification in which a methoxyethyl group replaces the 2′ hydrogen atom of the deoxyribose moiety (or the hydrogen atom of the 2′ hydroxyl of the ribose moiety) of a nucleoside or nucleotide. ASOs containing 2′MOE modifications exhibit 1) improved nuclease resistance, making them less likely to be degraded before they can hybridize to their target sequence; 2) increased affinity for their target sequence, making them more likely to exert their desired activity; and 3) lower toxicity, improving the safety of such ASOs for use in therapeutic applications. In some embodiments, at least 50%, at least 60%, at least 70%, at least 80%, at least 90%, at least 95%, at least 97%, at least 98%, or at least 99% of the nucleotides of the antisense oligonucleotide comprise a 2′MOE modification. In some embodiments, each of the nucleotides of the antisense oligonucleotide comprises a 2′MOE modification. In some embodiments, at least at least 50%, at least 60%, at least 70%, at least 80%, at least 90%, at least 95%, at least 97%, at least 98%, or at least 99% of the nucleotides of the antisense oligonucleotide comprise both a 2′MOE modification and a phosphorothioate modification. In some embodiments, each of the nucleotides of the antisense oligonucleotide comprise both a 2′MOE modification and a phosphorothioate modification. In some embodiments, each of the internucleoside phosphates of the antisense oligonucleotide comprises a phosphorothioate modification. An antisense oligonucleotide in which each internucleoside phosphate comprises a phosphorothiothioate modification may comprise one or more terminal phosphates (e.g., a 5′ and/or 3′ terminal phosphate), and each terminal phosphate may be an unmodified phosphate or a phosphorothioate. In some embodiments, each of the nucleotides of the antisense oligonucleotide comprises a 2′-O-methoxyethyl modification, and each of the internucleoside phosphates of the antisense oligonucleotide comprises a phosphorothioate modification.

A 2′-O-methyl (2′OMe) modification is a chemical modification in which a methyl group replaces the 2′ hydrogen atom of the deoxyribose moiety (or the hydrogen atom of the 2′ hydroxyl of the ribose moiety) of a nucleoside or nucleotide. ASOs containing 2′OMe modifications exhibit 1) improved nuclease resistance, making them less likely to be degraded before they hybridize to their target sequence, and 2) increased affinity to their target sequence, making them more likely to exert their desired activity.

2′MOE modifications to ASOs prevent RNase H from hydrolyzing the target RNA, but tight binding of the ASO to the target RNA results in efficient interference with splicing of the RNA through steric hindrance. The bound ASO prevents the protein-RNA interactions required for splicing at the target sequence, and ASOs containing 2′MOE modifications may thus be especially useful for manipulating the balance of different splicing isoforms of the same RNA within a cell.

### Splicing Factors

In some aspects, the present disclosure provides antisense oligonucleotides that bind to mRNA encoding a splicing factor. The role of mRNA in gene expression is well understood in the art but is described here for clarity.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). A gene, in some embodiments, includes a promoter sequence, coding regions (e.g., exons), non-coding regions (e.g., introns), and regulatory regions (also referred to as regulatory sequences). A promoter is a nucleotide sequence to which RNA polymerase binds to initial transcription (e.g., ATG). Promoters are typically located directly upstream from (at the 5′ end of) a transcription initiation site. A coding sequence (or other nucleotide sequence) is considered to be operably linked to a promoter if that promoter regulates transcription of the coding sequence.

A gene is transcribed when RNA polymerase, using DNA as a template, forms a primary transcript comprising a precursor RNA, or pre-mRNA. A pre-mRNA is processed into a messenger RNA, or mRNA, through several steps, including the addition of a 5′ cap, cleavage of the 3′ sequence, polyadenylation, and RNA splicing. An mRNA is translated when a ribosome binds to the coding region and recruits transfer RNAs, or tRNAs, corresponding to the codons of the mRNA. A codon is a sequence comprising three nucleotides of a nucleic acid encoding a specific amino acid, and an anticodon is a sequence comprising three nucleotides on a tRNA that hybridize with a codon. Each tRNA carries an amino acid that corresponds to its anticodon (e.g., the codon UCC encodes serine, and tRNAs with the anticodon AGG carry serine). Successive recruitment of tRNAs to corresponding codons by ribosomes results in the formation of a polypeptide, as amino acids are joined by dehydration synthesis, resulting in covalent bonds between the carbon atom of one amino acid and the nitrogen atom of another.

Transcription of a coding sequence by RNA polymerase results in the formation of a precursor mRNA, or pre-mRNA, which must be modified prior to translation. Pre-mRNAs contain a combination of exons, or coding regions, introns, or non-coding regions, which must be removed by RNA splicing. RNA splicing is a process in which introns (non-coding regions), are removed from a pre-mRNA, and exons (coding regions) are joined together, resulting in the formation of an mRNA that can be translated into an encoded protein. RNA splicing is mediated by the spliceosome, an RNA-protein complex containing multiple small nuclear riboproteins (snRNPs), which consist of small nuclear RNAs (snRNAs) and proteins, which may include survival of motor neuron (SMN) proteins and Gem-associated (Gemin) proteins. Assembly of snRNPs may also require other associated proteins, including U2 small nuclear RNA auxiliary factor (U2AF35), U2AF2 (U2AF65), and splicing factor 1 (SF1).

Biochemically, RNA splicing involves two transesterification reactions, which are well understood in the art. First, the 2′ hydroxyl of a specific branchpoint nucleotide within the intron forms a bond with the first nucleotide of the intron via nucleophilic attack, releasing the 3′ end of the first exon. Second, the 3′ hydroxyl of the released first exon performs an electrophilic attack on the last nucleotide of the intron, resulting in the joining of the first exon to the second, and the release of the intron. Alternatively, the 3′ hydroxyl of the first exon may bond to the last nucleotide of a different intron, resulting in the first exon being joined to the exon that follows that intron, and the release of all introns and exons between the two joined exons. This process is known as alternative RNA splicing, a process in which differential splicing of the same pre-mRNA results in the formation of different mRNA isoforms that encode different polypeptides.

Alternative RNA splicing is a key step in gene expression regulation and a rich source of proteomic and functional diversity in eukaryotes. Regulation of alternative splicing is essential for normal development and defects in splicing are implicated in many human diseases, including cancer. Alternative splicing is regulated by the core splicing machinery along with regulatory splicing factors (SFs) that bind specific RNA sequences and act in a dose-dependent manner. SFs are frequently altered in human diseases, leading to downstream changes in the spliced isoform repertoire.

In some embodiments, the antisense oligonucleotide binds to an mRNA encoding a serine-arginine rich (SR) splicing factor. In some embodiments, the antisense oligonucleotide binds to an mRNA encoding TRA2β. Serine-arginine rich (SR) proteins are a family of fourteen (14) essential SFs (SRSF1 to SRSF12, and SR-like proteins TRA2α and TRA2β) that evolved from a common ancestor. SR proteins contain at least one RNA recognition motif (RRM), a domain responsible for their binding to specific pre-mRNA sequences, and at least one serine-arginine rich domain that coordinates SR protein localization and protein interactions. SR proteins act at multiple steps of the spliceosome cycle, and are involved in alternative RNA splicing, promoting either exon skipping or inclusion. In addition, several SR proteins have splicing-independent roles, including mRNA export, mRNA decay, or translation regulation. Changes in SR protein levels can therefore affect a wide network of downstream RNA targets and alternative splicing events. During normal development, SR proteins are critical for maintenance of stem cell pluripotency, early embryo patterning, and proper development of mature tissues such as brain, heart, and liver. Additionally, defects in SR proteins have been causatively implicated in several pathologies, including cardiac and liver dysfunction, brain and developmental abnormalities, diabetes, lupus, and cancer.

Solid tumors frequently exhibit altered expression of SR proteins, which subsequently promotes the formation of RNA isoforms that impact key aspects of tumor biology. The molecular mechanisms driving alterations in SR protein levels in disease are poorly understood. Altered SR protein expression in tumors frequently occurs without underlying copy number changes in the genome, suggesting that dysregulation occurs at the transcriptional and post-transcriptional level. Transcriptional activation of SFs, for example by the Myc oncogene, has been observed in a subset of human tumors.

At the post-transcriptional level, SR proteins are regulated by alternative splicing coupled to nonsense-mediated decay (AS-NMD), an RNA surveillance mechanism predicted to regulate up to a third of human genes. Many SF genes contain regions ultraconserved during evolution that are predicted to undergo AS-NMD and trigger SF auto-regulation. In mRNAs encoding SR proteins, these ultraconserved regions contain either noncoding exons, also called “poison exons” (PE), or 3′ UTR poison sequences, that when included or spliced, respectively, introduce a premature termination codon and target the mRNA encoding the SR protein for auto-regulation via AS-NMD. For example, binding of the TRA2β protein to the TRA2β-PE sequence on the mRNA encoding TRA2β enhances inclusion of the TRA2β-PE in the mature mRNA, resulting in decay of the mRNA encoding TRA2β. Similar auto-regulatory patterns that directly link PE splicing and changes in protein levels have been demonstrated experimentally for five other SR proteins. In addition to auto-regulation, where an SR protein promotes inclusion of the PE in mRNA encoding the same SR protein, cross-regulation has also been observed, wherein an SR protein promotes the inclusion of PEs in mRNAs encoding other SFs. For example, TRA2β also promotes the inclusion of a PE in mRNAs encoding TRA2a, decreasing levels of TRA2α protein. Additionally, SRSF3 promotes inclusion of PEs in mRNAs encoding SRSF5 and SRSF7, thereby decreasing expression of SRSF5 and SRSF7 proteins. However, the full extent of cross-regulatory interactions within the SR protein family, and the sequence determinants of cross-regulation, are poorly understood.

In some embodiments, introduction of the antisense oligonucleotide into a cell increases inclusion of the TRA2β-PE inclusion on TRA2β-encoding mRNAs by at least 30%, at least 40%, at least 50%, at least 60%, at least, 70%, at least 80%, at least 90%, at least 95%, at least 97%, at least 98%, or at least 99% relative to introduction of a non-targeting control antisense oligonucleotide. In some embodiments, introduction of the antisense oligonucleotide into a cell decreases TRA2β protein expression by at least 30%, at least 40%, at least 50%, at least 60%, at least, 70%, at least 80%, at least 90%, at least 95%, at least 97%, at least 98%, or at least 99% relative to introduction of a non-targeting control antisense oligonucleotide.

### Sequence Identity and Nucleotide Substitutions

The present disclosure, in some embodiments, provides antisense oligonucleotides that bind to a target sequence, wherein the antisense oligonucleotide or target sequence comprises a sequence having at least 90% sequence identity to a reference sequence (e.g., SEQ ID NO: 2 or SEQ ID NO: 3). The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. An exemplary “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In some embodiments, the antisense oligonucleotide or target sequences have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence, or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned. In some embodiments, the antisense oligonucleotide or target sequence comprises at least 90%, at least 91%, at least 92%, at least 93%, at least 94%, at least 95%, at least 96%, at least 97%, at least 98%, or at least 99% sequence identity to a reference sequence.

In some embodiments, the antisense oligonucleotide comprises at least 90%, at least 91%, at least 92%, at least 93%, at least 94%, at least 95%, at least 96%, at least 97%, at least 98%, or at least 99% sequence identity to the nucleotide sequence of SEQ ID NO: 3. In some embodiments, the antisense oligonucleotide comprises at least 90% sequence identity to the nucleotide sequence of SEQ ID NO: 3. In some embodiments, the antisense oligonucleotide comprises at least 95% sequence identity to the nucleotide sequence of SEQ ID NO: 3. In some embodiments, the antisense oligonucleotide comprises at least 97% sequence identity to the nucleotide sequence of SEQ ID NO: 3. In some embodiments, the antisense oligonucleotide comprises at least 98% sequence identity to the nucleotide sequence of SEQ ID NO: 3. In some embodiments, the antisense oligonucleotide comprises at least 99% sequence identity to the nucleotide sequence of SEQ ID NO: 3. In some embodiments, the antisense oligonucleotide comprises the nucleotide sequence of SEQ ID NO: 3. In some embodiments, the antisense oligonucleotide comprises the nucleotide sequence of SEQ ID NO: 14.

In some embodiments, the antisense oligonucleotide binds to a target sequence comprising at least 90%, at least 91%, at least 92%, at least 93%, at least 94%, at least 95%, at least 96%, at least 97%, at least 98%, or at least 99% sequence identity to SEQ ID NO: 2. In some embodiments, the target sequence has at least 90% sequence identity to the nucleotide sequence of SEQ ID NO: 2. In some embodiments, the target sequence has at least 95% sequence identity to the nucleotide sequence of SEQ ID NO: 2. In some embodiments, the target sequence has at least 97% sequence identity to the nucleotide sequence of SEQ ID NO: 2. In some embodiments, the target sequence has at least 98% sequence identity to the nucleotide sequence of SEQ ID NO: 2. In some embodiments, the target sequence has at least 99% sequence identity to the nucleotide sequence of SEQ ID NO: 2. In some embodiments, the antisense oligonucleotide binds to a target sequence comprising the nucleotide sequence of SEQ ID NO: 2.

In other embodiments, an antisense oligonucleotide comprises a nucleotide substitution relative to the sequence of a reference sequence. A nucleotide substitution, with respect to a reference sequence, is a modification of a nucleic acid comprising the reference sequence, where a single nucleotide of the nucleic acid is replaced by another nucleotide with a different nucleic acid base, such that nucleic acid differs from the reference sequence at exactly one nucleotide position. A nucleic acid may comprise multiple nucleotide substitutions, such that at a nucleic acid with Z nucleotide substitutions relative to a references sequence comprises a sequence that differs from the reference sequence at exactly Z nucleotide positions. In some embodiments, an antisense oligonucleotide has no more than one, no more than two, no more than three, no more than four, or no more than five nucleotide substitutions relative to a reference sequence.

In some embodiments, the antisense oligonucleotide has 5 or fewer, 4 or fewer, 3 or fewer, 2 or fewer, or 1 or fewer nucleotide substitutions relative to SEQ ID NO: 3. In some embodiments, the antisense oligonucleotide comprises 5 nucleotide substitutions relative to SEQ ID NO: 3. In some embodiments, the antisense oligonucleotide comprises 4 nucleotide substitutions relative to SEQ ID NO: 3. In some embodiments, the antisense oligonucleotide comprises 3 nucleotide substitutions relative to SEQ ID NO: 3. In some embodiments, the antisense oligonucleotide comprises 2 nucleotide substitutions relative to SEQ ID NO: 3. In some embodiments, the antisense oligonucleotide comprises 1 nucleotide substitution relative to SEQ ID NO: 3.

In some embodiments, the antisense oligonucleotide binds to a target sequence comprising 5 or fewer, 4 or fewer, 3 or fewer, 2 or fewer, or 1 or fewer nucleotide substitutions relative to SEQ ID NO: 2. In some embodiments, the antisense oligonucleotide binds to a target sequence comprising 5 substitutions relative to SEQ ID NO: 2. In some embodiments, the antisense oligonucleotide binds to a target sequence comprising 4 substitutions relative to SEQ ID NO: 2. In some embodiments, the antisense oligonucleotide binds to a target sequence comprising 3 substitutions relative to SEQ ID NO: 2. In some embodiments, the antisense oligonucleotide binds to a target sequence comprising 2 substitutions relative to SEQ ID NO: 2. In some embodiments, the antisense oligonucleotide binds to a target sequence comprising 1 substitution relative to SEQ ID NO: 2.

In some embodiments, the antisense oligonucleotide is 10-15, 15-20, 20-25, 25-30, or 30-35, 35-40, 40-45, or 45-50 nucleotides in length. In some embodiments, the antisense oligonucleotide is 25 nucleotides in length. In some embodiments, the antisense oligonucleotide is 30 nucleotides in length. In some embodiments, the antisense oligonucleotide is 35 nucleotides in length. In some embodiments, the antisense oligonucleotide is 40 nucleotides in length.

### Delivery Vectors

The present disclosure provides, in some aspects, delivery vectors, optionally viral vectors, comprising an antisense oligonucleotide. A delivery vector refers to a composition that comprises a second composition to be delivered to a desired location. In some embodiments, a vector is an extra-chromosomal nucleic acid molecule capable of replication in a cell and to which an insert sequence can be operatively linked so as to bring about replication of the insert sequence. Examples include, but are not limited to, circular DNA molecules such as plasmids constructs, phage constructs, and cosmid vectors, as well as linear nucleic acid constructs (e.g., lambda phage constructs, and bacterial artificial chromosomes (BACs)). A vector may include expression signals such as a promoter and/or a terminator, a selectable marker such as a gene conferring resistance to an antibiotic, and one or more restriction sites into which insert sequences can be cloned. Vectors can have other unique features (such as the size of DNA insert they can accommodate). In some embodiments, the delivery vector is a viral vector. A viral vector is a virus or viral genome comprising RNA or DNA that comprises a sequence having at least 90% sequence identity to the reference sequence to be delivered. The use of viral vectors to deliver antisense oligonucleotides may alleviate the challenges posed by instability and poor cellular uptake of naked nucleic acid, resulting in increased antisense oligonucleotide activity due to the ability of the viral vector to both protect the nucleic acid and direct its delivery to the target cell. Non-limiting examples of viral vectors include retroviruses, lentiviruses, adenoviruses, and adeno-associated viruses (AAVs). Retroviruses and lentiviruses are members of the Retroviridae family with single-stranded RNA genomes which, upon entering a cell, produce a double-stranded DNA intermediate that integrates into the host genome, from which a sequence may be expressed. Adenoviruses are members of the Adenoviriade family with double-stranded DNA genomes. Adenoviruses do not integrate into the host genome, posing a lower risk of genotoxicity, and have been tested in gene therapy applications, such as the delivery of an SMN2-antisense U7 snRNA to promote the inclusion of SMN2 intron 7 to restore full-length SMN for the treatment of spinal muscular atrophy (SMA) (Geib et al. PLoS ONE. (2009). 4:e8204.). Adeno-associated viruses are members of the Parvoviridae family with single-stranded DNA genomes. They efficiently transduce cells of several tissues and display long-term efficacy in post-mitotic tissues, making them good candidates for gene transfer applications. Clinical trials using recombinant AAV (rAAV) vectors have shown promising results, including the treatment of familial lipoprotein lipoprotein lipase deficiency (Gaudet et al. Gene Ther. (2013). 20:361-369). Additionally, rAAVs encoding antisense oligonucleotides have shown promising preclinical results for the treatment of myotonic dystrophy type 1 (Furling et al. Gene Ther. (2003). 10:795-802) and is currently being developed further for clinical use (Bisset et al. Hum. Mol. Genet. (2015). 24:4971-4983). See also, Imbert et al. Genes. (2017). 8(2):51.

Delivery vectors are useful for delivering a desired composition, in this case an antisense oligonucleotide. Following administration to a subject, a nucleic acid vector may serve as a template for DNA replication, RNA transcription, or virus production by cellular machinery, which generate more copies of the desired nucleic acid. Delivery vectors may also protect the antisense oligonucleotide from being prematurely targeted, modified, or degraded by enzymes, small molecules, or other cellular components, thereby increasing the likelihood that the antisense oligonucleotide will exert the desired activity. Viral vectors, when formulated as a virus containing DNA or RNA comprising a desired sequence, may be engineered to target delivery of the virus, viral vector, and/or antisense oligonucleotide to a desired cell type or location, such as by designing a virus with surface proteins that promote delivery of viral RNA or DNA to a cell type expressing a surface receptor that interacts with the viral surface protein. Both nucleic acid vectors and viral vectors comprising a desired sequence to be expressed may optionally comprise a promoter operably linked to the desired sequence, and the promoter may be chosen such that the desired sequence is expressed under only desired conditions, such as in the presence or absence of a given environmental stimulus associated with a certain cell or tissue type. Methods of engineering viruses for targeted delivery to desired cells or tissues, and the selection or promoters for context-dependent expression of a desired sequence are well understood by one of ordinary skill in the art.

### Pharmaceutical Compositions and Methods of Use

The antisense oligonucleotides provided herein may be formulated in a pharmaceutical composition comprising an antisense oligonucleotide and a pharmaceutically acceptable excipient. A pharmaceutically acceptable excipient can also be incorporated in a formulation and can be any excipient (e.g., carrier) known in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

Any pharmaceutically acceptable excipients are known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, and silicylate. Each pharmaceutically acceptable excipients used in a pharmaceutical composition of the invention must be “acceptable” in the sense of being compatible with the other ingredients of the formulation and not injurious to the subject. Excipients suitable for a selected dosage form and intended route of administration are well known in the art, and acceptable diluents or carriers for a chosen dosage form and method of administration can be determined using ordinary skill in the art.

In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA (e.g., for transplantation into a subject), hyaluronidase, nanoparticle mimics and combinations thereof.

In some embodiments, the antisense oligonucleotides provided herein are formulated in a delivery vehicle, which may be a cationic lipid nanoparticle (LNP). A lipid nanoparticle is a small particle, often with dimensions in the 1*10⁻⁹ m to 1*10⁻⁷ m range, comprising lipids that surround an agent to be delivered, in this case an antisense oligonucleotide. Lipid nanoparticles typically comprise ionizable cationic lipid, non-cationic lipid, sterol and PEG lipid components along with the nucleic acid cargo to be delivered. In some embodiments, the antisense oligonucleotides provided herein are formulated in a delivery vehicle, which may be a liposome. A liposome is a spherical vesicle comprising at least one lipid bilayer surrounding the agent to be delivered, in this case an antisense oligonucleotide. A liposome may contain one or more lipids, such as phosphatidylcholine and phosphatidylethanolamine, but may contain any lipids that can form a lipid bilayer. Enclosure of nucleic acids in lipid nanoparticles helps protect them from degradation by nucleases and prolongs their persistence in vivo, thereby increasing their efficacy. LNPs have shown promise in the delivery of ASOs targeting proprotein convertase subtilisin/kexin type 9 (PCSK9) mRNA in preclinical studies in mice (Yang et al. Mol. Ther. Nucleic Acids. (2020). 19:1357-1367), superoxide dismutase I (SOD1) in vitro and in zebrafish for the treatment of amyotrophic lateral sclerosis (Chen et al. Front. Neurosci. (2017). 11:476), and B-cell lymphoma 2 (Bcl-2) for the treatment of lung cancer (Cheng et al. Pharm. Res. (2017). 34:310-320). Lipid nanoparticles can be generated using components, compositions, and methods generally known in the art, for example PCT/US2013/023359, PCT/US2014/061793, PCT/US2015/034496, PCT/US2016/052352, PCT/US2017/061113, all of which are incorporated by reference herein in their entirety.

Routes of administration of the antisense oligonucleotides provided herein include, for example, intravenous, intramuscular, intraperitoneal, subcutaneous, intranasal, and intratumoral.

Thus, in some embodiments, a composition comprising an antisense oligonucleotide may be formulated for intravenous, intramuscular, intraperitoneal, subcutaneous, intranasal, or intratumoral delivery.

The present disclosure provides, in some embodiments, a kit comprising an antisense oligonucleotide, vector, or pharmaceutical composition, along with a delivery device. A delivery device is an object that may be used for administering a composition to a subject or environment. Non-limiting examples of delivery devices include needles, syringes, drug cartridges, nasal spray canisters, and transdermal patches. In some embodiments, the kit is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acids of the pharmaceutical composition are relatively stable over time.

The present disclosure, in some aspects, provides methods of administering antisense oligonucleotides or compositions comprising antisense oligonucleotides to a subject (e.g., a human subject) with cancer. Cancer refers to a physiological condition in mammals that is typically characterized by unregulated cell growth or proliferation. Non-limiting examples of cancer include lymphoma, leukemia, sarcoma, blastoma, melanoma, renal/kidney cancer, testicular cancer, prostate cancer, thyroid cancer, pancreatic cancer, lung cancer, liver cancer, esophageal cancer, cervical cancer, uterine cancer, ovarian cancer, stomach cancer, colon cancer, bladder cancer, and breast cancer, such as triple-negative breast cancer.

In some embodiments, the subject has breast cancer, which may be triple-negative breast cancer. Breast cancer refers to a cancer characterized by unregulated growth, replication, and/or proliferation of breast cells, which may include cells of the ducts, lobules, or connective tissue of the breast or mammary tissue. Breast cancers are typically characterized by the expression profile of three surface markers or surface receptors: estrogen receptor, progesterone receptor, and human epidermal growth factor receptor 2 (HER2). Triple-negative breast cancer (TNBC) is a breast cancer in which cells express none of these three surface markers, and so therapeutic approaches targeted towards estrogen receptor, progesterone receptor, and/or HER2 have diminished or no activity in TNBC-negative cells.

In some embodiments, the subject has ovarian cancer. In some embodiments, the subject has colon cancer. In some embodiments, the subject has glioblastoma, In some embodiments, the subject has bladder cancer. In some embodiments, the subject has kidney cancer. In some embodiments, the subject has liver cancer. In some embodiments, the subject has lung cancer. In some embodiments, the subject has prostate cancer.

In some embodiments of the methods provided herein, administration of the antisense oligonucleotide to a subject results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the volume of a tumor in the subject. In some embodiments, the method results in a reduction of at least 10% of the volume of the tumor in the subject. In some embodiments, the method results in a reduction of at least 30% of the volume of the tumor in the subject. See, e.g., Weber, J Nucl Med. 50(Suppl 1):1S-10S.

In some embodiments, administration of the antisense oligonucleotide to a subject results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of one or more tumors in the subject. In some embodiments, the method results in a reduction of at least 30% of the mass of one or more tumors in the subject.

In some embodiments, administration of the antisense oligonucleotide to a subject results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of a primary tumor in the subject. In some embodiments, the method results in a reduction of at least 30% of the mass of a primary tumor in the subject. In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the volume of a primary tumor in the subject. In some embodiments, the method results in a reduction of at least 30% of the volume of a primary tumor in the subject. In some embodiments, a primary tumor refers to the largest tumor in a subject, by mass or volume. In some embodiments, a primary tumor refers to a tumor in which the largest percentage of cells are cancer cells.

In some embodiments, administration of the antisense oligonucleotide to a subject results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the tumor growth rate in the subject. In some embodiments, the method results in a reduction of at least 10% of the tumor growth rate in the subject. In some embodiments, the method results in a reduction of at least 30% of the tumor growth rate in the subject. See, e.g., Hather et al., Cancer Inform. 2014. 13(Suppl 4):

### EXAMPLES

### Example 1. Inducing TRA2β-PE Inclusion with Splice-Switching Antisense Oligonucleotides Increases Cell Death and Decreases Proliferation and Cell Motility in Breast Cancer Models

Regulation of splicing-factor (SF) expression determines the balance of different RNA isoforms within a cell, and dysregulation of SF levels is associated with diseases such as cancer. SFs contain ultraconserved poison exon (PE) sequences, which exhibit greater identity across species than nearby coding exons, yet their physiological role and molecular regulation is incompletely understood. Splicing factors that are dysregulated in diseases such as cancer thus present a promising therapeutic target. Splice-switching antisense RNA oligonucleotides (ASOs), a class of FDA-approved RNA-based therapeutics that bind to reverse complementary regions on target pre-mRNA (FIGS. 1A and 2A), have successfully been used to alter splicing in human diseases by blocking regulatory sequences (Lieberman, 2018). Here, the potential of splice-switching ASOs, including a novel ASO with modified backbone chemistry, to inhibit TRA2β protein expression was investigated.

### Identification of Regulatory Regions for Targeting by ASOs

Previous minigene experiments identified exonic and intronic regions that impact TRA2β-PE splicing that could be targeted using ASOs (FIG. 3). A computational method that incorporates RNA secondary structure and SF binding motifs (Tabaglio et al., 2018) was used to design six ASOs targeting the TRA2β ultraconserved region and predicted to promote either inclusion or skipping. Endogenous TRA2β-PE splicing and TRA2β protein expression were examined in MDA-MB231 or HS578T breast cancer cell lines transfected with TRA2β-targeting or non-targeting control 2′-O-methyl (2′OMe) phosphorothioated (PS) ASOs. Two ASOs decreased TRA2β-PE inclusion and increased protein expression (2′OMe ASO-1565 and 2′OMe ASO-1567), and two increased exon inclusion and decreased protein expression (2′OMe ASO-1566 and 2′OMe ASO-1570) compared to non-targeting control (2′OMe ASO-CTL).

2′OMe ASO-1570, which had a strong effect on TRA2β-PE inclusion in minigene experiments, targets a downstream intronic region that contains predicted intronic splice silencer motifs and predicted hnRNPA1 binding sites (FIG. 2A). Additionally, 2′OMe ASO-1570 overlaps a minigene deletion mutant (423) that enhanced TRA2β-PE inclusion (FIG. 2A), suggesting these sites are functionally important for regulating TRA2β-PE inclusion. Furthermore, overexpression of hnRNPA1-CDS along with the TRA2β-PE minigene decreased TRA2β-PE inclusion and increased TRA2β protein expression (FIG. 2D). Finally, co-transfection of an increasing amount of hnRNPA1-CDS along with 2′OMe ASO-1570 can off-compete the ASO splice-switching effects and increase TRA2β protein levels (FIG. 2E), suggesting that ASO-1570 functions, at least in part, by blocking hnRNPA1 intronic silencer motifs.
Development of 2′MOE ASO-1570 with 2′-O-Methoxyethyl and Phosphorothioate Chemical Modifications

The effect of ASO-mediated inclusion of TRA2β-PE on cancer-relevant phenotypes was assayed in vitro using a novel chemically modified ASO based on the sequence of 2′OMe ASO-1570, which exhibited the strongest splicing switch activity but limited effects on TRA2β protein expression (FIGS. 2B-C). This novel ASO contained the same sequence as 2′OMe ASO-1570, but no 2′OMe modifications, instead containing 2′-O-methoxyethyl (2′MOE) backbone modifications. This ASO is referred to below as “2′MOE ASO-1570” or “ASO-1570,” while the control ASO with non-targeting sequence but the same 2′MOE and PS modifications to the backbone is referred to as “2′MOE ASO-CTL” or “ASO-CTL.” The sequences of 2′MOE ASO-1570 and 2′MOE ASO-CTL, which were investigated further, are shown in Table 1.

TABLE 1
Sequences of 2′MOE antisense oligonucleotides.

| ASO | | |
|---|---|---|
| Name | Target Sequence | ASO sequence |
| ASO- | Non-targeting | TCATTTGCTTCATACAGG (SEQ ID |
| CTL | | NO: 1) |
| ASO- | TAGAGGAAAATATATACAGGGTA | AAGGCACTTCTTTACCCTGTATA |
| 1570 | AAGAAGTGCCTT (SEQ ID NO: 2) | TATTTTCCTCTA (SEQ ID NO: 3) |

### Effect of 2′MOE ASO-1570 on TRA213-PE Splicing, Cell Proliferation, Cell Death, and Motility in Breast Cancer Models

The 2′MOE ASO-1570 increased TRA2β-PE inclusion and subsequently decreased TRA2β protein levels by −30-50% when transfected into MDA-MB231, SUM159, or HS578T breast cancer cells (FIGS. 1B and 2F). Breast cancer cells treated with 2′MOE ASO-1570 exhibited decreased cell proliferation as measured by 5-Ethynyl-2′-deoxyuridine (EdU) incorporation (FIG. 1C), and increased cell death as measured using Annexin V and Hoechst stains (FIG. 1D) compared to 2′MOE ASO-CTL. The strongest effects were detected in MDAMB231 cells which also exhibited the largest splicing switch and TRA2β protein KD (FIGS. 1B-1D) compared to 2′MOE ASO-CTL. 2′MOE ASO-1570-treated HS578T cells displayed increased cell death, but no changes in cell proliferation at the 48 h time point (FIGS. 2G-H), possibly because of their slower growth rate. The effect of 2′MOE ASO-1570 on SUM159 and HS578T cell migration was measured in wound healing assays, excluding MDA-MB231 cells which exhibit a strong cell death phenotype (FIG. 1D) that would be a confounding factor when quantifying cell migration. 2′MOE ASO-1570 decreased wound closure in both SUM159 and HS578T cells compared to 2′MOE ASO-CTL, which likely reflects a reduction in both the migratory and proliferative potential of these cancer cells (FIG. 2I). Interestingly, even a small change in TRA2β-PE inclusion (ΔPSI<10%) in SUM159 or HS578T cells resulted in −30% decrease of TRA2β protein levels and impacted both cell death and migration (FIGS. 1D and 2H-I).

Given the observation that 2′MOE ASO-1570 decreases TRA2β protein levels, its effects on the splicing of TRA2β target transcripts were examined to determine whether this ASO exhibits on-target effects. Exon inclusion was quantified in a set of six known TRA2/3 splicing targets, previously identified in MDA-MB231 and SUM159 cells with shRNA-mediated TRA2β protein KD (Park et al., 2019). MDA-MB231 cells treated with 2′MOE ASO-1570 exhibited increased inclusion in GOLGAS exon 24, PFKM exon 10 and TRA2α-PE (exon 3), as well as increased skipping in AZIN1 exon 3, IFI44 exon 2 and KIF23 exon 18 compared to 2′MOE ASO-CTL (FIG. 1E). This pattern mimics the splicing changes detected in MDA-MB231 cells with TRA2β-targeting vs. control shRNAs (Park et al., 2019), validating 6 out of 6 TRA2β-regulated splicing events in this cell line. Similarly, 5 out of 6 of these events were validated in both SUM159 and HS578T (FIGS. 1E and 2J), again closely following the patterns of TRA2β-targeting shRNA which, similarly to 2′MOE ASO-1570, did not alter PFKM splicing in these cell lines (Park et al., 2019). Here again, the magnitude of ASO-mediated splicing changes across the three cell lines was proportional to the ASO-mediated change in TRA2β-PE inclusion and protein expression (FIGS. 1B and 2F).

Next, the effect of 2′MOE ASO-1570 on two non-transformed mammary epithelial cell lines was examined. Similar to its effects in breast cancer cells, 2′MOE ASO-1570 increased TRA2β-PE inclusion and decreased TRA2β protein expression in both MCF-10A and AR7-HMEC cells compared to 2′MOE ASO-CTL (FIG. 1F). However, 2′MOE ASO-1570-treated non-transformed cells did not exhibit increased cell death (FIG. 1G) or decreased cell migration (FIG. 2K) compared to cells transfected with 2′MOE ASO-CTL. GFP-tagged MDA-MB231 cells were then co-cultured together with MCF-10A cells and simultaneously transfected with 2′MOE ASO-1570 or 2′MOE ASO-CTL. The proportion of GFP-tagged MDA-MB231 cells to total cells was measured by fluorescent imaging at different timepoints after ASO transfection. Starting at day 5, the percent of MDA-MB231 cells decreased in the 2′MOE ASO-1570 vs. 2′MOE ASO-CTL treated populations (FIG. 2L), suggesting that breast cancer cells are more sensitive to changes in TRA2β protein levels compared to non-transformed mammary epithelial cells. To determine the effects of TRA2β-PE inclusion across multiple cancer types, sequencing data from tumor samples in The Cancer Genome Atlas (TCGA) were analyzed using a computational pipeline that utilizes rMATS to predict exon inclusion from RNA-sequencing reads. Correlations between TRA2β-PE inclusion and patient survival were analyzed for each tumor type. Patients with low TRA2β-PE inclusion had lower survival probabilities across several tumor types, including breast, cervical, acute myeloid lymphoma (AML), lung, and skin cancers (FIG. 4). Thus, decreased inclusion of the TRA2β poison exon (PE) is associated with lower overall survival across multiple cancer types.

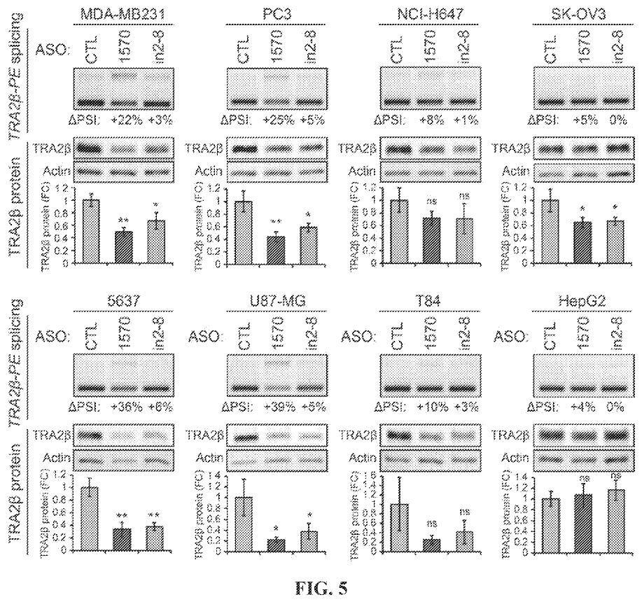

To determine the ability of ASOs such as ASO-1570 to modulate TRA2β poison exon inclusion across cancer subtypes, ASO-CTL, ASO-1570, and ASO-in2-8 ASOs were transfected separately into triple negative breast cancer MDA-MB231, prostate cancer PC3, lung cancer NCI-H647, ovarian cancer SK-OV3, bladder cancer 5637, glioblastoma U87-MG, colon cancer T84, and liver cancer HepG2 human cell lines. Each ASO contained 2′MOE-modified nucleotides and PS backbone modifications. ASO-1570 promoted increased poison exon inclusion and decreased TRA2β protein levels across multiple cell lines (FIG. 5), with the strongest effects in glioblastoma, bladder, colon, breast, prostate, and ovarian cancer models. ASO-in2-8 exhibited similar effects but was less efficient compared to ASO-1570. Thus, 2′MOE ASO-1570 modulates splicing of TRA2β poison exon and decreases TRA2β protein levels across multiple cancer subtypes.

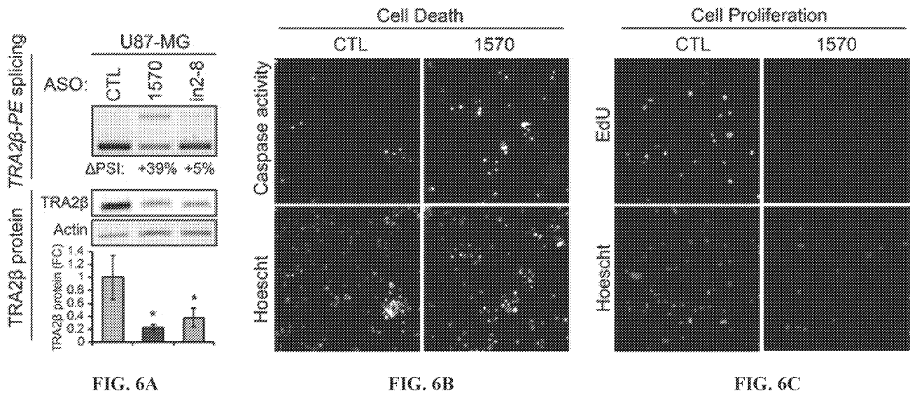

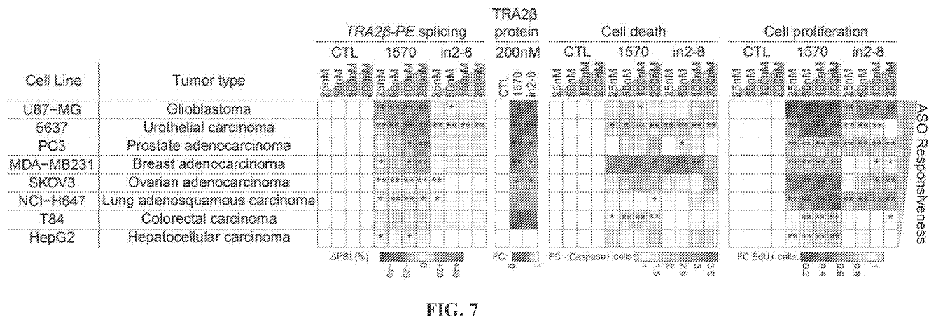

Next, dose-dependent effects of 2′MOE ASO-1570, and ASO-in2-8 transfections were assayed in triple negative breast cancer MDA-MB231, prostate cancer PC3, lung cancer NCI-H647, ovarian cancer SK-OV3, bladder cancer 5637, glioblastoma U87-MG, colon cancer T84, and liver cancer HepG2 human cell lines. Assays measured effects on mRNA splicing and translation (FIG. 6A), cell death (FIG. 6B), and cell proliferation (FIG. 6C).

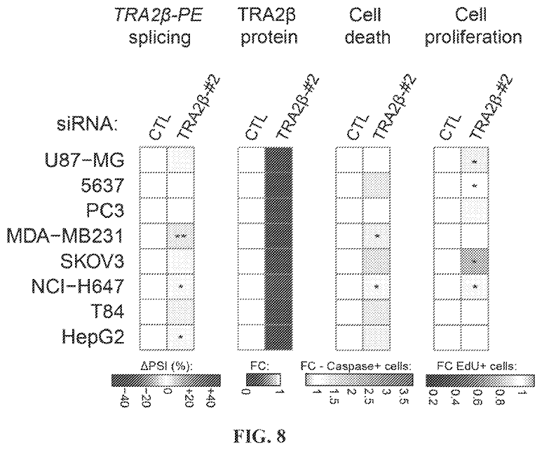

2′MOE ASO-1570 impacted TRA2β mRNA splicing in a dose-dependent manner across all cell models tested (FIG. 8). 2′MOE ASO-1570 also promoted increased cell death and decreased cell proliferation across multiple cancer models, with the strongest effects in glioblastoma, bladder, colon, breast, prostate, and ovarian cancer models (FIG. 8). Thus, 2′MOE ASO-1570 exhibited pro-tumoricidal activity across multiple cancer subtypes.

To define the mechanisms of action of ASO-1570, its effects were compared to siRNA-mediated TRA2β protein knockdown (FIG. 8). TRA2β-targeting siRNAs decreased TRA2β protein levels but did not impact cell death or cell proliferation of various cancer cell lines (FIG. 9). Thus, the effects of 2′MOE ASO-1570 are distinct from TRA2β-targeting siRNA, suggesting that the anti-cancer effects of 2′MOE ASO-1570 do not rely solely on decreased protein TRA2β levels.

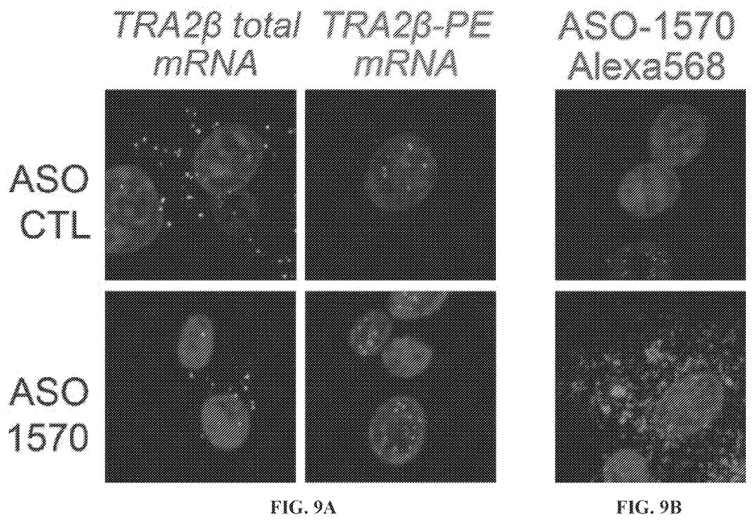

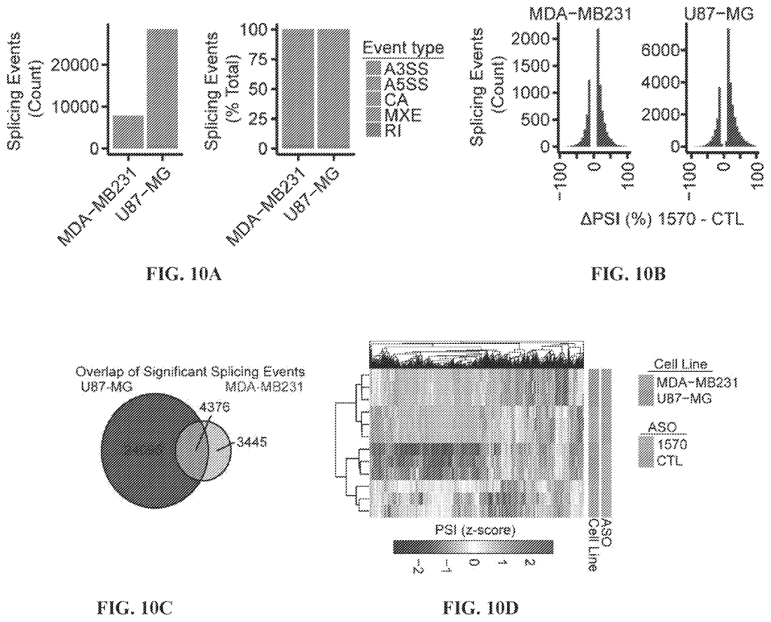

To evaluate the contributions of cellular localization of ASOs and TRA2β transcripts to the effects of ASOs such as ASO-1570, the locations of TRA2β poison exon-containing transcripts were evaluated in cells treated with ASO-CTL or ASO-1570 using RNA FISH probes specific to the poison-exon, to the coding transcript, or to both (FIG. 10A). Each ASO contained 2′MOE-modified nucleotides and PS backbone modifications. Cells treated with ASO-1570 exhibited increased abundances of TRA2β poison exon-containing transcript in the nucleus, and decreased abundances of TRA2β coding transcript in the cytoplasm, suggesting that ASO-1570 promotes increased expression of the TRA2β poison exon-containing transcripts in the nucleus, Moreover, use of Alexa-labeled ASO-1570 demonstrated that only a fraction of the ASO localized in the nucleus following transfection into cells (FIG. 10B), suggesting that even small doses of the ASO are sufficient to achieve therapeutic effects.

The impact of 2′MOE ASO-1570 transfection on alternative splicing was evaluated in U87-MG glioblastoma cells and MDA-MB231 triple-negative breast cancer cells through RNA-sequencing analysis. Both cell lines exhibited striking changes in RNA isoform expression, with thousands of differential splicing events between ASO-1570 and ASO-CTL treatment (FIGS. 11A-11B). While these cancer cells are derived from drastically different tissues of origin, they exhibited a strong overlap in differential splicing events between ASO-1570 and ASO-CTL (FIGS. 11C-11D), suggesting common on-target effects across multiple tumor types.

Taken together, these data suggest that targeting PE splicing events in oncogenic SFs represents a viable strategy to decrease SF levels and limit the survival of SF-dependent cancer cells while leaving a therapeutic window to limit the effects on normal cells. 2′MOE ASO-1570, a novel antisense oligonucleotide with 2′-O-methoxyethyl and phosphorothioate backbone modifications, is one promising ASO that exhibits such therapeutic effects, including decreasing proliferation and inducing cell death, in SF-dependent cancer cells but not healthy cells.

### Materials and Methods

### Human Cell Lines

HEK293, HEK293T, and HeLa cell lines were obtained and maintained in DMEM (Gibco) supplemented with 10% FBS, 1% penicillin streptomycin (Sigma). MDA-MB231 shRNA-TRA2β (Park et al., 2019), SUM159, and HS578T cells were maintained in DMEM (Gibco) supplemented with 20% FBS, 1% penicillin streptomycin (Sigma). MCF-10A (ATCC) were maintained in DMEM/F12 supplemented with 5% horse serum (Gibco), 100 ug/mL epidermal growth factor, 1 mg/mL hydrocortisone, 1 mg/mL cholera toxin, 10 mg/mL insulin, and 1% penicillin streptomycin (Sigma). HMEC AR-7 were grown in mammary epithelial cell basal medium (PromoCell) supplemented with 0.4% bovine pituitary extract, 10 ng/mL epidermal growth factor, 5 ug/mL insulin, 0.5 ug/mL hydrocortisone All cell lines were grown at 37° C. under a humidified atmosphere with 5% CO2. Cells were routinely tested negative for mycoplasma using the MycoAlert™ Mycoplasma Detection Kit (Lonza), and cell aliquots from early passages were used. All cell lines used here were established from female subjects.

### ASO Transfections

24 h prior to transfection, MDA-MB231, SUM159 or HS578T cells were seeded into a 12 well plate at 250,000 cells per well. Cells were transfected using lipofectamine 3000 with i) 50 nM of 2′-O-methyl-phosphorothioated (2′OMe and PS-modified backbones) TRA2β-targeting (TechNOA tNOA-2′OMe-hTRA2B-01 #1565, hTRA2B-01 #1566, hTRA2B-01 #1567, hTRA2B-02 #1568, hTRA2B-02 #1569, hTRA2B-02 #1570; alternatively 2′OMe ASO-1565, 2′OMe ASO-1566, 2′OMe ASO-1567, 2′OMe ASO-1568, 2′OMe ASO-1569, and 2′OMe ASO-1570) or non-targeting control (TechNOA tNOA-2′OMe-NC, alternatively 2′OMe ASO-CTL) ASOs; or ii) 50 nM of 2′-O-methoxyethyl-phosphorothioated TRA2β-targeting ASO-1570 (2′MOE ASO-1570) (IDT) (Table 1) or non-targeting control ASO (2′MOE ASO-CTL) (Sahashi et al., 2012). All ASOs were purified using RNAse-free high performance liquid chromatography. Cells were collected 48 h after transfection by lifting with 2 mM EDTA in PBS and analyzed by RT-PCR and western blot.

### RT-PCR Analysis

Cells were harvested as described above and RNA was extracted using an RNAeasy kit (Qiagen) including DNAse I treatment. 500-1000 ng of RNA was reverse transcribed using Superscript III reverse transcriptase (Invitrogen). Semi-quantitative PCR was used to amplify cDNA with Phusion hot start II DNA polymerase (Thermo Fisher) and primers listed in Tables 2-3. PCR products were separated in 1% or 2% agarose gel stained with SYBR Safe (Invitrogen) and imaged using ChemiDoc MP Imaging System (Bio-rad). PCR bands were quantified using ImageLab 6.0 software (Bio-rad) and the percent spliced in (PSI) ratio of each exon-containing transcript was calculated as the exon-included isoform band intensity divided by the intensity of included and skipped isoform bands. ΔPSI is calculated as PSIcase-PSIcontrol (HA-empty vector for minigene experiments, non-targeting gRNA for CASFx experiments, non-targeting ASO-CTL for ASO experiments).

TABLE 2
RT-PCR primers used for semi-quantitative PCR of TRA2B splicing targets.

| Target | Forward Primer and Sequence | Reverse Primer and Sequence |
|---|---|---|
| Endogenous | TRA2A-5UTR-F | TRA2A-Ex5-R |
| TRA2A | TTGCCGACTCTTTCCTCTTCC | GGATCTGGATCGAGTGTAACGT |
| | (SEQ ID NO: 4) | CTA (SEQ ID NO: 5) |
| Endogenous | IFI44-F | IFI44-R |
| IFI44 | ATCCAGACAGAGCAGCTAC | GGCAGACAGTAAGCTCTTCC |
| | C (SEQ ID NO: 6) | (SEQ ID NO: 7) |
| Endogenous | PFKM-F | PFKM-R |
| PFKM | ATGACGACTGGGAGGAACA | CACTACACAGGCTGGGGTAT |
| | C (SEQ ID NO: 8) | (SEQ ID NO: 9) |
| Endogenous | GOLGA4-F | GOLGA4-R |
| GOLGA4 | CCACCGTACTGAAGTTCCCT | CTGCAACTTATCTGTGGGCC |
| | (SEQ ID NO: 10) | (SEQ ID NO: 11) |
| Endogenous | AZIN1-F | AZIN1-R |
| AZIN1 | AGAGGGCCTAGGAGAAGTG | CATCTCAGCCGTATTCCACA |
| | C (SEQ ID NO: 12) | (SEQ ID NO: 13) |

### Western Blot Analysis

Cells were harvested as described above and lysed in Laemmli buffer (50 mM Tris-HCl pH 6.2, 5% (v/v) β-mercaptoethanol, 10% (v/v) glycerol, 3% (w/v) SDS). Cell lysate was run on 8-16% gradient gels (Biorad), transferred onto 0.2 urn nitrocellulose membranes (Millipore) and blocked in 5% (w/v) milk in Tween 20-TBST (50 mM Tris pH 7.5, 150 mM NaCl, 0.05% (v/v) Tween 20). Blots were incubated with anti-Myc Tag (Cell Signaling #2276), anti-HA Tag (Cell Signaling #3724), anti-β Tubulin (GenScript #A01203-40), anti-TRA2β (Abcam #ab31353), anti-GAPDH (GenScript #A01622). Anti-mouse IR-Dye 680 or IR-Dye 800 conjugated anti-mouse or rabbit IgG secondary antibodies (LICOR) were used for infrared detection with a ChemiDoc MP Imaging System (Bio-rad). Protein expression was quantified using ImageLab 6.0 software (Bio-rad) and normalized first to a lane loading control and then expressed as fold change (FC) or Log 2FC to experimental controls.

### Immunofluorescence

48 hours after transfection with 500 ng of pCI-neo-HA-SR-CDS or pMAX-CASFx-SR, HeLa cells were fixed using 5% formalin in PBS, washed with IF buffer (7.6 g/L NaCl, 1.896 g/L Na2HPO4, 0.414 g/L NaH2PO4, 0.5 g/L NaN3, 1 g/L BSA, 0.2% Triton X-100, Tween-20, pH 7.4), permeabilized with 0.5% TritonX-100, and blocked with 10% goat serum (Sigma). HA primary antibody (Cell signaling #3724) was used at 1:500 dilution and Alexa-fluor-568 conjugated secondary antibody (Invitrogen) was used at 1:500 dilution. Imaging was performed on Ti Eclipse Widefield Nikon fluorescent microscope.

### Cell Death Assays

48 h after 2′MOE ASO transfections, MDA-MB231, SUM159, and HS578T cells were incubated with AnnexinV-Alexa647 (Invitrogen; 1:100) and Hoechst (Life Technologies; 5 ng/mL) in 1× AnnexinV binding buffer (Invitrogen; 10 mM HEPES, 140 mM NaCl, 2.5 mM CaCl2, pH 7.4) for 15 minutes at 37° C. Pre-warmed media was added to wells and plates were imaged with using a Phenix high content confocal imaging system (Perkin Elmer). Four fields of view per replicate were captured using a 10× objective. AnnexinV+ and total Hoescht+ cells were counted using the Columbus analysis software (Perkin Elmer). Data is represented as fold change in AnnexinV+/Total cells between targeting and control ASO treated samples.

### Cell Proliferation Assays

24 h prior to transfection with 2′MOE ASOs, MDA_MB231, SUM159 or HS578T cells were seeded into a 48 well plate at 50,000 cells per well. 48 h after 2′MOE ASO transfections, MDA-MB231, SUM159, or HS578T cells were incubated with 10 μM EdU for 24 h. EdU labelling was detected using the Click-iT cell proliferation kit (Thermo Fisher C10340). Briefly, cells were fixed in 5% formalin then permeabilized using 0.5% 34riton-100. EdU was detected with an alexa-647 azide, and total cell DNA was stained with Hoescht 33342 (5 μg/mL). Plates were imaged with using a Phenix high content confocal imaging system (Perkin Elmer). 25 fields of view per replicate were captured using a 10× objective. EdU+ and total Hoescht+ cells were counted using the Columbus analysis software (Perkin Elmer). Data is represented as percentage of EdU+ cells for both ASO-1570 and the control ASO.

### Wound Closure Assays

24 h prior to transfection with 2′MOE ASOs, SUM159 and HS578T cells were seeded into a 48 well plate at 50,000 cells per well. 48 h after transfection the cell monolayer was scratched with a P200 tip, then washed with PBS and refreshed with culture media. Plates were imaged with brightfield using a Phenix high content confocal imaging system and a 10× objective (Perkin Elmer) at 0 h, 12 h, 24 h, and 48 h after the initial scratch. To obtain a representative image of the scratch, four fields of view were stitched together using a 10% overlap on the Phenix Harmony system and exported for analysis in ImageJ. Wound closure was measured using the MRI wound healing tool plugin for ImageJ. All measurements were normalized to the initial scratch area (0 h measurement) for each replicate to generate a percent wound closure.

Co-culture assay 24 h prior to transfection with 2′MOE ASOs, MCF-10A and EGFP expressing MDA-MB231 cells were seeded together into a 48 well plate at 30,000 and 20,000 cells, respectively, in MCF-10A growth media. Prior to imaging, cells were stained with Hoechst (Life Technologies; 5 ng/mL). Plates were imaged at 0 h (day of transfection), 2 d, 3 d, 5 d, 10 d, and 15 d after transfection using a Phenix high content confocal imaging system and a 10× objective (Perkin Elmer). Cell segmentation and counting on the EGFP channel was done using the Phenix Harmony system and used to quantify the number of MDA-MB231 cells in the culture. Total cell populations per well were quantified by counting Hoechst+ nuclei using the Phenix Harmony system. Proportion of MDA-MB231 cells to total cells in each well was quantified by dividing the number of EGFP+ cells by total Hoechst+ cells.

### Quantification and Statistical Analysis

Where indicated, data is presented as the mean±standard deviation. For RT-PCR, western blot, immunofluorescence quantifications, and wound healing significant differences to a control are assessed using an unpaired two-tailed Student t-test.

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

What is claimed is:

1. An antisense oligonucleotide comprising a sequence that binds to a target sequence on an mRNA that encodes TRA2β, wherein the antisense oligonucleotide further comprises a 2′-O-methoxyethyl modification.

2. The antisense oligonucleotide of claim 1, wherein at least 80% of the nucleotides of the antisense oligonucleotide comprise a 2′-O-methoxyethyl modification.

3. The antisense oligonucleotide of claim 1 or 2, wherein each of the nucleotides of the antisense oligonucleotide comprises 2′-O-methoxyethyl modification.

4. The antisense oligonucleotide of any one of claims 1-3, further comprising a phosphorothioate modification.

5. The antisense oligonucleotide of claim 4, wherein at least 80% of the internucleoside phosphates of the antisense oligonucleotide comprise a phosphorothioate modification.

6. The antisense oligonucleotide of claim 4 or 5, wherein each of the internucleoside phosphates of the antisense oligonucleotide comprise a phosphorothioate modification.

7. The antisense oligonucleotide of any one of claims 1-3, wherein each of the nucleotides of the antisense oligonucleotide comprises a 2′-O-methoxyethyl modification, and each of the internucleoside phosphates of the antisense oligonucleotide comprises a phosphorothioate modification.

8. The antisense oligonucleotide of any one of claims 1-7, wherein the target sequence is within an intronic splicing silencer sequence.

9. The antisense oligonucleotide of any one of claims 1-8, wherein the target sequence comprises a sequence having at least 90% sequence identity to the sequence of SEQ ID NO: 2.

10. The antisense oligonucleotide of claim 9, wherein the target sequence comprises a sequence having at least 95% sequence identity to the sequence of SEQ ID NO: 2.

11. The antisense oligonucleotide of claim 10, wherein the target sequence comprises the sequence of SEQ ID NO: 2.

12. The antisense oligonucleotide of any one of claims 1-11, wherein the oligonucleotide comprises a sequence having at least 90% sequence identity to the sequence of SEQ ID NO: 3.

13. The antisense oligonucleotide of claim 12, wherein the oligonucleotide comprises a sequence having at least 95% sequence identity to the sequence of SEQ ID NO: 3.

14. The antisense oligonucleotide of any one of claims 1-13, comprising no more than two nucleotide substitutions relative to the sequence of SEQ ID NO: 3.

15. The antisense oligonucleotide of any one of claims 1-14, comprising no more than one nucleotide substitution relative to the sequence of SEQ ID NO: 3.

16. The antisense oligonucleotide of claim any one of claims 1-15, wherein the oligonucleotide comprises the sequence of SEQ ID NO: 3.

17. The antisense oligonucleotide of any one of claims 1-16, wherein binding of the antisense oligonucleotide increases TRA2β-PE inclusion and/or decreases TRA2β protein expression by at least 30% relative to a non-targeting control antisense oligonucleotide.

18. The antisense oligonucleotide of claim 1-17 formulated in a delivery vehicle.

19. The antisense oligonucleotide of claim 18, wherein the delivery vehicle is a cationic lipid nanoparticle.

20. The antisense oligonucleotide of claim 18, wherein the delivery vehicle is a liposome.

21. A delivery vector comprising the antisense oligonucleotide of any one of claims 1-12.

22. The delivery vector of claim 21, wherein the delivery vector is a viral vector.

23. A host cell comprising the antisense oligonucleotide of any one of claims 1-20 or the delivery vector of claim 21 or 22.

24. The host cell of claim 23, wherein the host cell is a cancer cell, optionally a breast cancer cell, such as a triple-negative breast cancer cell, an ovarian cancer cell, a colon cancer cell, a glioblastoma cell, a bladder cancer cell, a kidney cancer cell, a liver cancer cell, a lung cancer cell, or a prostate cancer cell.

25. A pharmaceutical composition comprising the antisense oligonucleotide of any one of claims 1-20, or the delivery vector of claim 21, and a pharmaceutically acceptable excipient.

26. A method comprising administering to a subject the antisense oligonucleotide of any one of claims 1-20 or the pharmaceutical composition of claim 25.

27. The method of claim 26, wherein the subject has breast cancer, ovarian cancer, colon cancer, glioblastoma, bladder cancer, kidney cancer, liver cancer, lung cancer, or prostate cancer.

28. The method of claim 27, wherein the subject has triple-negative breast cancer.

29. The method of any one of claims 26-28, wherein the administering is intravenous, intramuscular, intraperitoneal, subcutaneous, intranasal, or intratumoral.

30. A method comprising synthesizing the antisense oligonucleotide of any one of claims 1-20.

31. A method comprising formulating the antisense oligonucleotide of any one of claims 1-20 with a pharmaceutically acceptable excipient.

32. A kit comprising the antisense oligonucleotide of any one of claims 1-20 or the pharmaceutical composition of claim 25, and a delivery device.


SEQUENCE LISTING (17 sequences)

SEQ ID NO: 1 - DNA, 18 bp, Artificial Sequence
  Synthetic
tcatttgcttcatacagg

SEQ ID NO: 2 - DNA, 35 bp, Artificial Sequence
  Synthetic
tagaggaaaatatatacagggtaaagaagtgcctt

SEQ ID NO: 3 - DNA, 35 bp, Artificial Sequence
  Synthetic
aaggcacttctttaccctgtatatattttcctcta

SEQ ID NO: 4 - DNA, 21 bp, Artificial Sequence
  Synthetic
ttgccgactctttcctcttcc

SEQ ID NO: 5 - DNA, 25 bp, Artificial Sequence
  Synthetic
ggatctggatcgagtgtaacgtcta

SEQ ID NO: 6 - DNA, 20 bp, Artificial Sequence
  Synthetic
atccagacagagcagctacc

SEQ ID NO: 7 - DNA, 20 bp, Artificial Sequence
  Synthetic
ggcagacagtaagctcttcc

SEQ ID NO: 8 - DNA, 20 bp, Artificial Sequence
  Synthetic
atgacgactgggaggaacac

SEQ ID NO: 9 - DNA, 20 bp, Artificial Sequence
  Synthetic
cactacacaggctggggtat

SEQ ID NO: 10 - DNA, 20 bp, Artificial Sequence
  Synthetic
ccaccgtactgaagttccct

SEQ ID NO: 11 - DNA, 20 bp, Artificial Sequence
  Synthetic
ctgcaacttatctgtgggcc

SEQ ID NO: 12 - DNA, 20 bp, Artificial Sequence
  Synthetic
agagggcctaggagaagtgc

SEQ ID NO: 13 - DNA, 20 bp, Artificial Sequence
  Synthetic
catctcagccgtattccaca

SEQ ID NO: 14 - DNA, 35 bp, Artificial Sequence; identical to SEQ ID NO: 3
  Synthetic
  modified_base (1)..(35): modified by 2′-methoxyethyl
  modified_base (2)..(35): modified by phosphorothioate

SEQ ID NO: 15 - DNA, 51 bp, Artificial Sequence
  Synthetic
ttatttgtttaatagaggaaaatatatacagggtaaagaagtgccttccag

SEQ ID NO: 16 - DNA, 50 bp, Artificial Sequence
  Synthetic
gaagaaggaaaatgcggaagtcgtcatttgacaagttttataaatgagta

SEQ ID NO: 17 - DNA, 50 bp, Artificial Sequence
  Synthetic
aatgcatgctaattatcagaccagaagtcccacttgtagaatattgagca